(19) United States
(12) Patent Application Publication
(10) Pub. No.: US 2010/0197590 A1
(43) Pub. Date: Aug. 5, 2010

(54) 4-AMINO-7,8-DIHYDROPYRIDO[4,3-d]PYRIMIDIN-5(6H)-ONE DERIVATIVES

(75) Inventors: Robert L. Dow, Groton, CT (US); Michael J. Munchhof, Salem, CT (US)

Correspondence Address: PFIZER INC.;PATENT DEPARTMENT, Bld 114 M/S 9114, EASTERN POINT ROAD, GROTON, CT, 06340, US

(73) Assignee: Pfizer Inc

(21) Appl. No.: 12/697,701

(22) Filed: Feb. 1, 2010

Related U.S. Application Data
(60) Provisional application No. 61/149,375, filed on Feb. 3, 2009.
(60) Provisional application No. 61/149,522, filed on Feb. 3, 2009.
(60) Provisional application No. 61/285,258, filed on Dec. 10, 2009.

(51) Int. Cl. A61K 38/16; A61K 31/702; A61K 31/55; A61K 31/519; C07D 471/04; A61P 3/04; A61P 3/10

(52) U.S. Cl. 514/12; 514/42; 514/217.01; 514/264.11; 544/279

(57) ABSTRACT

The invention provides compounds of Formula (Ia)

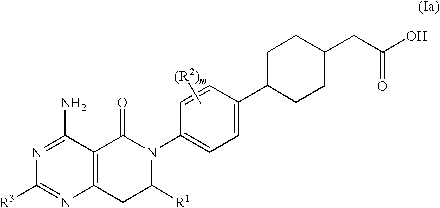

or pharmaceutically acceptable salts thereof, as well as the preparation, compositions and uses thereof, where R¹, R², R³, and m are defined as described above.

This application claims priority from U.S. Provisional Application No. 61/149,375, filed on Feb. 3, 2009; U.S. Provisional Application No. 61/149,522, filed on Feb. 3, 2009 and U.S. Provisional Application No. 61/285,258, filed on Dec. 10, 2009.

### FIELD OF THE INVENTION

The invention relates to 4-amino-7,8-dihydropyrido[4,3-d]pyrimidin-5(6 h)-one derivatives including pharmaceutical compositions and uses thereof.

### BACKGROUND

It is estimated that somewhere between 34 and 61 million people in the US are obese and, in much of the developing world, incidence is increasing by about 1% per year. Obesity increases the likelihood of death from all causes by 20%, and more specifically, death from coronary artery disease and stroke are increased by 25% and 10%, respectively. Key priorities of anti-obesity treatments are to reduce food intake and/or hyperlipidemia. Since the latter has been suggested to provoke insulin resistance, molecules developed to prevent the accumulation of triglyceride would not only reduce obesity but they would also have the additional effect of reducing insulin resistance, a primary factor contributing to the development of diabetes. The therapeutic activity of leptin agonists has come under scrutiny through their potential to reduce food intake and, also, to reverse insulin resistance; however, their potential may be compromised by leptin-resistance, a characteristic of obesity. Acyl coenzyme A:diacylglycerol acyltransferase 1 (DGAT-1) is one of two known DGAT enzymes that catalyze the final step in mammalian triglyceride synthesis and an enzyme that is tightly implicated in both the development of obesity and insulin resistance. DGAT-1 deficient mice are resistant to diet-induced obesity through a mechanism involving increased energy expenditure. US researchers have now shown that these mice have decreased levels of tissue triglycerides, as well as increased sensitivity to insulin and to leptin. Importantly, DGAT-1 deficiency protects against insulin resistance and obesity in agouti yellow mice, a model of severe leptin resistance. Thus, DGAT-1 may represent a useful target for the treatment of insulin and leptiri resistance and hence human obesity and diabetes. Chen, H. C., et al., J Clin Invest, 109(8), 1049-55 (2002).

Although studies show that DGAT-1 inhibition is useful for treating obesity and diabetes, there remains a need for DGAT-1 inhibitors that have efficacy for the treatment of metabolic disorders (e.g., obesity, Type 2 diabetes, and insulin resistance syndrome (also referred to as “metabolic syndrome”)).

### SUMMARY OF THE INVENTION

Compounds of the invention include those represented by Formula (Ia):

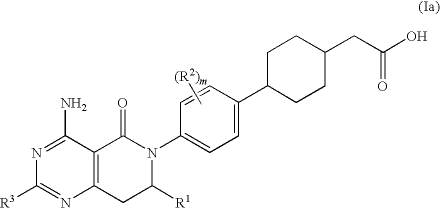

or a pharmaceutically acceptable salt thereof, in which R¹ is hydrogen (C₁-C₂)alkoxy, halo-substituted (C₁-C₂)alkyl, halo-substituted (C₁-C₂)alkoxy) or (C₁-C₂)alkyl; each R² is independently halogen, OH, (C₁-C₄)alkyl, cyano, (C₃-C₆)cycloalkyl or (C₁-C₄)alkoxy; R³ is hydrogen, (C₁-C₂)alkyl, (C₁-C₂)alkoxy, or —O(C₁-C₂)alkyl (C₁-C₂)alkoxy; and m is 0, 1, 2, or 3.

Another aspect of the invention is a pharmaceutical composition that comprises: (1) a compound of the invention, and (2) a pharmaceutically acceptable excipient, diluent, or carrier. The composition may comprise a therapeutically effective amount of a compound of the invention. The composition may also contain at least one additional pharmaceutical agent. Such agents include, for example, anti-obesity agents and/or anti-diabetic agents.

In yet another aspect of the invention, a method for treating a disease, disorder, or condition modulated by DGAT-1 inhibition in animals is provided that includes the step of administering to an animal, such as a human, in need of such treatment a therapeutically effective amount of a compound of the invention (or a pharmaceutical composition thereof). Diseases, conditions, and/or disorders mediated by DGAT-1 inhibition include, e.g., obesity (including weight control or weight maintenance), Type 2 diabetes, diabetic nephropathy, insulin resistance syndrome, hyperglycemia, hyperinsulinemia, hyperlipidemia, impaired glucose tolerance, hypertension, and reducing the level of blood glucose.

Compounds of the invention may be administered in combination with other pharmaceutical agents (in particular, anti-obesity and anti-diabetic agents including those described herein below). The combination therapy may be administered as (a) a single pharmaceutical composition which comprises a compound of the invention, at least one additional pharmaceutical agent described herein and a pharmaceutically acceptable excipient, diluent, or carrier; or (b) two separate pharmaceutical compositions comprising (i) a first composition comprising a compound of the invention and a pharmaceutically acceptable excipient, diluent, or carrier, and (ii) a second composition comprising at least one additional pharmaceutical agent described herein and a pharmaceutically acceptable excipient, diluent, or carrier. The pharmaceutical compositions may be administered simultaneously or sequentially and in any order.

It is to be understood that both the foregoing summary and the following detailed description and attendant claims are exemplary and explanatory only and are not restrictive of the invention, as claimed.

### DETAILED DESCRIPTION

The invention may be understood even more readily by reference to the following detailed description of exemplary embodiments of the invention and the examples included therein.

It is to be understood that this invention is not limited to specific synthetic methods of making that may of course vary. It is also to be understood that the terminology used herein is for the purpose of describing particular embodiments only and is not intended to be limiting. The plural and singular should be treated as interchangeable, other than the indication of number.

The headings within this document are only being utilized to expedite its review by the reader. They should not be construed as limiting the invention or claims in any manner.

In this specification and in the claims that follow, reference will be made to a number of terms that shall be defined to have the following meanings:

As used herein in the specification, “a” or “an” may mean one or more. As used herein in the claim(s), when used in conjunction with the word “comprising”, the words “a” or “an” may mean one or more than one. As used herein “another” may mean at least a second or more.

The term “about” refers to a relative term denoting an approximation of plus or minus 10% of the nominal value it refers, in one embodiment, to plus or minus 5%, in another embodiment, to plus or minus 2%. For the field of this disclosure, this level of approximation is appropriate unless the value is specifically stated require a tighter range.

As used herein, the term “alkyl” refers to a hydrocarbon radical of the general formula CₙH₂ₙ₊₁. The alkane radical may be straight or branched. For example, the term “(C₁-C₆)alkyl” refers to a monovalent, straight, or branched aliphatic group containing 1 to 6 carbon atoms (e.g., methyl, ethyl, n-propyl, i-propyl, n-butyl, i-butyl, s-butyl, t-butyl, n-pentyl, 1-methylbutyl, 2-methylbutyl, 3-methylbutyl, neopentyl, 3,3-dimethylpropyl, hexyl, 2-methylpentyl, and the like). Similarly, the alkyl portion (i.e., alkyl moiety) of an alkoxy group has the same definition as above. “Halo-substituted alkyl” or “halo-substituted alkoxy” refers to an alkyl or alkoxy group substituted with one or more halogen atoms (e.g., fluoromethyl, difluoromethyl, trifluoromethyl, perfluoroethyl, 1,1-difluoroethyl and the like).

The term “cycloalkyl” refers to nonaromatic rings that are fully hydrogenated and may exist as a single ring, bicyclic ring or a spiral ring. Unless specified otherwise, the carbocyclic ring is generally a 3- to 6-membered ring. For example, cycloalkyl include groups such as cyclopropyl, cyclobutyl, cyclopentyl, cyclohexyl, cyclohexenyl, and the like.

“Halogen” or “halo” refers to refers to a chlorine, fluorine, iodine, or bromine atom.

The phrase “therapeutically effective amount” means an amount of a compound of the invention that (i) treats or prevents the particular disease, condition, or disorder, (ii) attenuates, ameliorates, or eliminates one or more symptoms of the particular disease, condition, or disorder, or (iii) prevents or delays the onset of one or more symptoms of the particular disease, condition, or disorder described herein.

The term “animal” refers to humans (male or female), companion animals (e.g., dogs, cats and horses), food-source animals, zoo animals, marine animals, birds and other similar animal species. “Edible animals” refers to food-source animals such as cows, pigs, sheep and poultry.

The phrase “pharmaceutically acceptable” indicates that the substance or composition must be compatible chemically and/or toxicologically, with the other ingredients comprising a formulation, and/or the mammal being treated therewith.

The terms “treating”, “treat”, or “treatment” embrace both preventative, i.e., prophylactic, and palliative treatment.

The terms “modulated” or “modulating”, or “modulate(s)”, as used herein, unless otherwise indicated, refers to the inhibition of the diacylglycerol O-acyltransferase 1 (DGAT-1) enzyme with compounds of the invention.

The terms “mediated” or “mediating” or “mediate(s)”, as used herein, unless otherwise indicated, refers to the treatment or prevention the particular disease, condition, or disorder, (ii) attenuation, amelioration, or elimination of one or more symptoms of the particular disease, condition, or disorder, or (iii) prevention or delay of the onset of one or more symptoms of the particular disease, condition, or disorder described herein, by inhibiting the DGAT-1 enzyme.

The terms “compounds (or compound) of the invention” or simply “compounds” or “compound” (unless specifically identified otherwise) refer to compounds encompassed within this application, such as compounds encompassed within general formulas and intermediates of the compounds as well as salts, all stereoisomers (including diastereoisomers and enantiomers), tautomers, conformational isomers, and isotopically labeled compounds. Hydrates and solvates of the compounds are considered to be part of the invention, wherein the compound is in association with water or solvent, respectively.

The term “salt” and “pharmaceutically acceptable salt” refers to inorganic and organic salts of a compound. These salts can be prepared in situ during the final isolation and purification of a compound, or by separately reacting the compound with a suitable organic or inorganic acid or base and isolating the salt thus formed. Representative salts include the hydrobromide, hydrochloride, hydroiodide, sulfate, bisulfate, nitrate, acetate, trifluoroacetate, oxalate, besylate, palmitiate, pamoate, malonate, stearate, laurate, malate, borate, benzoate, lactate, phosphate, hexafluorophosphate, benzene sulfonate, tosylate, formate, citrate, maleate, fumarate, succinate, tartrate, naphthylate, mesylate, glucoheptonate, lactobionate, and laurylsulphonate salts, and the like. These may include cations based on the alkali and alkaline earth metals, such as sodium, lithium, potassium, calcium, magnesium, and the like, as well as non-toxic ammonium, quaternary ammonium, and amine cations including, but not limited to, ammonium, tetramethylammonium, tetraethylammonium, methylamine, dimethylamine, trimethylamine, triethylamine, ethylamine, and the like. See, e.g., Berge, et al., J. Pharm. Sci., 66, 1-19 (1977).

In one embodiment, the compounds are represented by Formula (Ib):

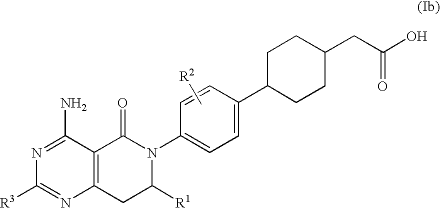

or a pharmaceutically acceptable salt thereof; in which R¹ is hydrogen or (C₁-C₂)alkyl; R² is hydrogen, halogen, OH, (C₁-C₄)alkyl, or (C₁-C₄)alkoxy; R³ is hydrogen, (C₁-C₄)alkyl or (C₁-C₂)alkoxy.

In one embodiment, R¹ is hydrogen or methyl and R³ is hydrogen, methyl or methoxy.

In another embodiment, compounds are represented by Formula (II):

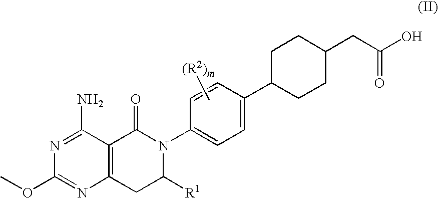

or a pharmaceutically acceptable salt thereof in which R¹ is hydrogen and R² is selected from hydrogen, halogen, OH, (C₁-C₄)alkyl, or (C₁-C₄)alkoxy.

In another embodiment, compounds are represented by Formula (III):

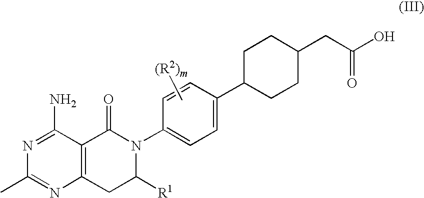

or a pharmaceutically acceptable salt thereof, in which R¹ is hydrogen and R² is hydrogen, halogen, OH, (C₁-C₄)alkyl, or (C₁-C₄)alkoxy.

In another embodiment, compounds are represented by Formula (IV):

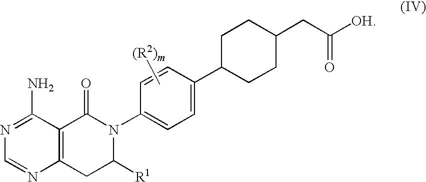

or a pharmaceutically acceptable salt thereof in which R¹ is hydrogen, and R² is hydrogen, halogen, (C₁-C₂)alkyl, or (C₁-C₂)alkoxy.

The invention also includes solvates and hydrates of the compounds of the invention. The term “solvate” refers to a molecular complex of a compound of this invention (including pharmaceutically acceptable salts thereof) with one or more solvent molecules. Such solvent molecules are those commonly used in the pharmaceutical art, which are known to be innocuous to the recipient, e.g., water, ethanol, ethylene glycol, and the like, The term “hydrate” refers to the complex where the solvent molecule is water. The solvates and/or hydrates may exist in crystalline form. Other solvents may be used as intermediate solvates in the preparation of more desirable solvates, such as methanol, methyl t-butyl ether, ethyl acetate, methyl acetate, (S)-propylene glycol, (R)-propylene glycol, 1,4-butyne-diol, and the like.

The compounds of the invention may contain asymmetric or chiral centers, and, therefore, exist in different stereoisomeric forms. Unless specified otherwise, it is intended that all stereoisomeric forms of the compounds of the invention as well as mixtures thereof, including racemic mixtures, form part of the invention. In addition, the invention embraces all geometric and positional isomers. For example, if a compound of the invention incorporates a double bond or a fused ring, both the cis- and trans-forms, as well as mixtures, are embraced within the scope of the invention.

Diastereomeric mixtures can be separated into their individual diastereoisomers on the basis of their physical chemical differences by methods well known to those skilled in the art, such as by chromatography and/or fractional crystallization. Enantiomers can be separated by converting the enantiomeric mixture into a diastereomeric mixture by reaction with an appropriate optically active compound (e.g., chiral auxiliary such as a chiral alcohol or Mosher's acid chloride), separating the diastereoisomers and converting (e.g., hydrolyzing) the individual diastereoisomers to the corresponding pure enantiomers. Also, some of the compounds of the invention may be atropisomers (e.g., substituted biaryls) and are considered as part of this invention. Enantiomers can also be separated by use of a chiral HPLC column. Alternatively, the specific stereoisomers may be synthesized by using an optically active starting material, by asymmetric synthesis using optically active reagents, substrates, catalysts or solvents, or by converting one stereoisomer into the other by asymmetric transformation.

It is also possible that the intermediates and compounds of the invention may exist in different tautomeric forms, and all such forms are embraced within the scope of the invention. The term “tautomer” or “tautomeric form” refers to structural isomers of different energies which are interconvertible via a low energy barrier. For example, proton tautomers (also known as prototropic tautomers) include interconversions via migration of a proton, such as keto-enol and imine-enamine isomerizations. A specific example of a proton tautomer is the imidazole moiety where the proton may migrate between the two ring nitrogens. Valence tautomers include interconversions by reorganization of some of the bonding electrons.

Certain compounds of the invention may exist in different stable conformational forms which may be separable. Torsional asymmetry due to restricted rotation about an asymmetric single bond, for example, because of steric hindrance or ring strain, may permit separation of different conformers.

The invention also embraces isotopically-labeled compounds of the invention which are identical to those recited herein, but for the fact that one or more atoms are replaced by an atom having an atomic mass or mass number different from the atomic mass or mass number usually found in nature. Examples of isotopes that can be incorporated into compounds of the invention include isotopes of hydrogen, carbon, nitrogen, oxygen, phosphorus, sulfur, fluorine, iodine, and chlorine, such as ²H, ³H, ¹¹C, ¹³C, ¹⁴C_{,} ¹³N, ¹⁵N, ¹⁵O, ¹⁷O, ¹⁸O, ³¹P, ³²P_{,} ³⁵S, ¹⁸F, ¹²³I, ¹²⁵I and ³⁸Cl, respectively.

Certain isotopically-labeled compounds of the invention (e.g., those labeled with ³H and ¹⁴C) are useful in compound and/or substrate tissue distribution assays. Tritiated (i.e., ³H) and carbon-14 (i.e., ¹⁴C) isotopes may be used for their ease of preparation and detectability. Further, substitution with heavier isotopes such as deuterium (i.e., ²H) may afford certain therapeutic advantages resulting from greater metabolic stability (e.g., increased in vivo half-life or reduced dosage requirements) and hence may be used in some circumstances. Positron emitting isotopes such as ¹⁸O, ¹³N, ¹¹C, and ¹⁸F are useful for positron emission tomography (PET) studies to examine substrate occupancy. Isotopically labeled compounds of the invention can generally be prepared by following procedures analogous to those disclosed in the Schemes and/or in the Examples herein below, by substituting an isotopically labeled reagent for a non-isotopically labeled reagent.

Certain compounds of the invention may exist in more than one crystal form (generally referred to as “polymorphs”). Polymorphs may be prepared by crystallization under various conditions, for example, using different solvents or different solvent mixtures for recrystallization; crystallization at different temperatures; and/or various modes of cooling, ranging from very fast to very slow cooling during crystallization. Polymorphs may also be obtained by heating or melting the compound of the invention followed by gradual or fast cooling. The presence of polymorphs may be determined by solid probe NMR spectroscopy, IR spectroscopy, differential scanning calorimetry, powder X-ray diffraction or such other techniques.

In general, compounds of this invention may be prepared by methods that include processes known in the chemical arts, particularly in light of the description contained herein in combination with the knowledge of the skilled artisan. Although other reagents, starting materials, intermediate compounds or methods can be used in practice or testing, generalized methods for the preparation of the compounds of the invention are illustrated by the following descriptions, Preparations, and reaction Schemes. Other preparation methods are described in the experimental section. The methods disclosed herein, including those outlined in the Schemes, Preparations, and Examples are for intended for illustrative purposes and are not to be construed in any manner as limitations thereon.

The starting materials are generally available from commercial sources such as Aldrich Chemicals (Milwaukee, Wis.) or are readily prepared using methods well known to those skilled in the art (e.g., prepared by methods generally described in Louis F. Fieser and Mary Fieser, Reagents for Organic Synthesis, v. 1-19, Wiley, New York (1967-1999 ed.), or Beilsteins Handbuch der organischen Chemie, 4, Aufl. ed. Springer-Verlag, Berlin, including supplements (also available via the Beilstein online database)).

Those skilled in the art will appreciate that other synthetic routes may be used to synthesize the inventive compounds. Although specific starting materials and reagents are depicted in the schemes and discussed below, other starting materials and reagents can be easily substituted to provide a variety of derivatives and/or reaction conditions. In addition, many of the compounds prepared by the methods described below can be further modified in light of this disclosure using conventional chemistry well known to those skilled in the art.

Compounds of the present invention may be synthesized by synthetic routes that include processes analogous to those well-known in the chemical arts, particularly in light of the description contained herein. The starting materials are generally available from commercial sources such as Aldrich Chemicals (Milwaukee, Wis.) or are readily prepared using methods well known to those skilled in the art (e.g., prepared by methods generally described in Louis F. Fieser and Mary Fieser, Reagents for Organic Synthesis, v. 1-19, Wiley, New York (1967-1999 ed.), or Beilsteins Handbuch der organischen Chemie, 4, Aufl. ed. Springer-Verlag, Berlin, including supplements (also available via the Beilstein online database)).

For illustrative purposes, the reaction schemes depicted below provide potential routes for synthesizing the compounds of the present invention as well as key intermediates. For a more detailed description of the individual reaction steps, see the Examples section below. Those skilled in the art will appreciate that other synthetic routes may be used to synthesize the inventive compounds. Although specific starting materials and reagents are depicted in the schemes and discussed below, other starting materials and reagents can be easily substituted to provide a variety of derivatives and/or reaction conditions. In addition, many of the compounds prepared by the methods described below can be further modified in light of this disclosure using conventional chemistry well known to those skilled in the art.

In the preparation of compounds of the present invention, protection of remote functionality (e.g., primary or secondary amine) of intermediates may be necessary. The need for such protection will vary depending on the nature of the remote functionality and the conditions of the preparation methods. Suitable amino-protecting groups (NH-Pg) include acetyl, trifluoroacetyl, t-butoxycarbonyl (BOC), benzyloxycarbonyl (CBz) and 9-fluorenylmethyleneoxycarbonyl (Fmoc). The need for such protection is readily determined by one skilled in the art. For a general description of protecting groups and their use, see T. W. Greene, Protective Groups in Organic Synthesis, John Wiley & Sons, New York, 1991.

Scheme I outlines the general procedures one could use to provide compounds of Formula (II).

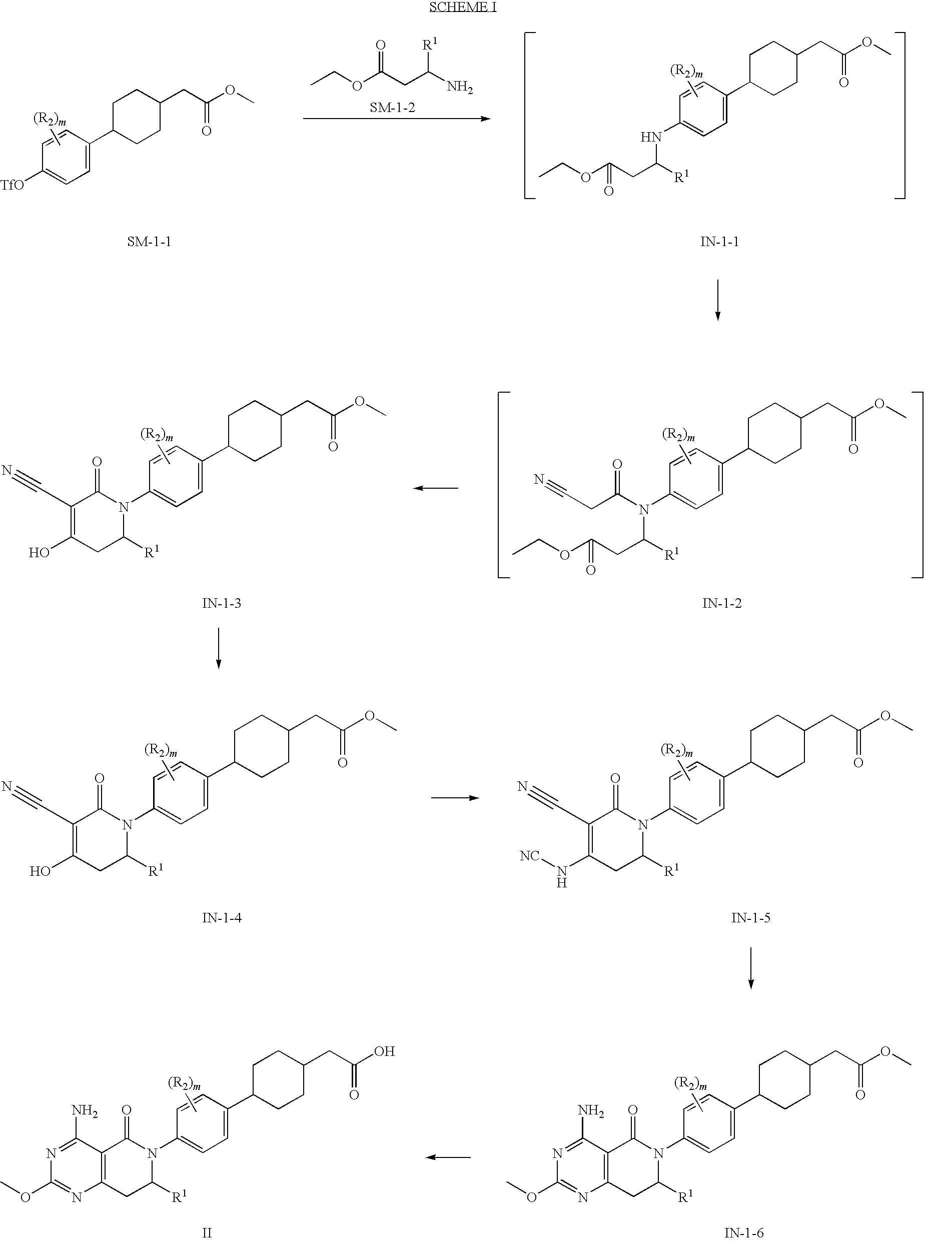

The desired starting material (SM-1-1) may be prepared as described in US Application No. 2004/0209871 for Compound 56, or prepared using analogous procedures. Starting materials (SM-1-2) such as, ethyl 3-aminopropanoate, i.e. where R¹ is H, may be purchased from commercial sources as the hydrochloride salt. Compounds where R¹ is methyl, i.e. 3-amino-butyric acid ethyl ester, may also be purchased from commercial sources as a racemic mixture or, if desired, as single enantiomers.

The two starting materials can be coupled together at elevated temperatures (e.g., about 80° C. to about 130° C.) in the presence of a Palladium (or copper) catalyst, a weak base (e.g., cesium carbonate), and 2-dicyclohexyl phosphino-2′,4′,6′-triisopropylbiphenyl (X-PHOS) in an inert environment to form intermediate (IN-1-1). Cyanoacetic acid is then added to the secondary amino group of intermediate (IN-1-1) via an amide coupling using procedures well known to those of skill in the art (e.g., addition of cyanoacetic acid in the presence of an activator such as N—N′-diisopropylcarbodiimide (DIC) and a mild base, such as 4-dimethylaminopyridine (DMAP) in an appropriate solvent such as acetonitrile (ACN) to form intermediate (IN-1-2). Formation of the lactam (IN-1-3) can be achieved by treatment with a base such as 1,8-diazabicycloundec-7-ene. (DBU) in methanol. The cyclization reaction may be carried out at elevated temperatures. Methylation of the lactam intermediate can be accomplished via the addition of oxalyl chloride in the presence of dichloromethane (DCM) and dimethylsulfoxide (DMSO) at low temperature followed by the addition of methanol. The resulting methoxy lactam intermediate (IN-1-4) can then be reacted with cyanamide in the presence of sodium methoxide and methanol to provide the corresponding aminonitrile intermediate (IN-1-5). A second cyclization reaction is affected via treatment with a strong mineral acid, e.g., sulfuric acid, in a protic solvent, e.g., methanol (MeOH), to form the aminopyrimidine intermediate (IN-1-6). This reaction may be conducted at elevated temperatures. The aminopyrimidine intermediate (IN-1-6) is a mixture of cis- and trans isomers which may be separated by chromatography using procedures well known to those of skill in the art. Formation of the corresponding carboxylic acid, a compound of Formula II, is accomplished by treatment with a strong base, such as potassium hydroxide (KOH) or sodium hydroxide (NaOH), in the presence of water and one or more polar solvents, e.g., MeOH and tetrahydrofuran (THF).

Scheme II outlines the general procedures one could use to provide compounds of the invention having Formula III.

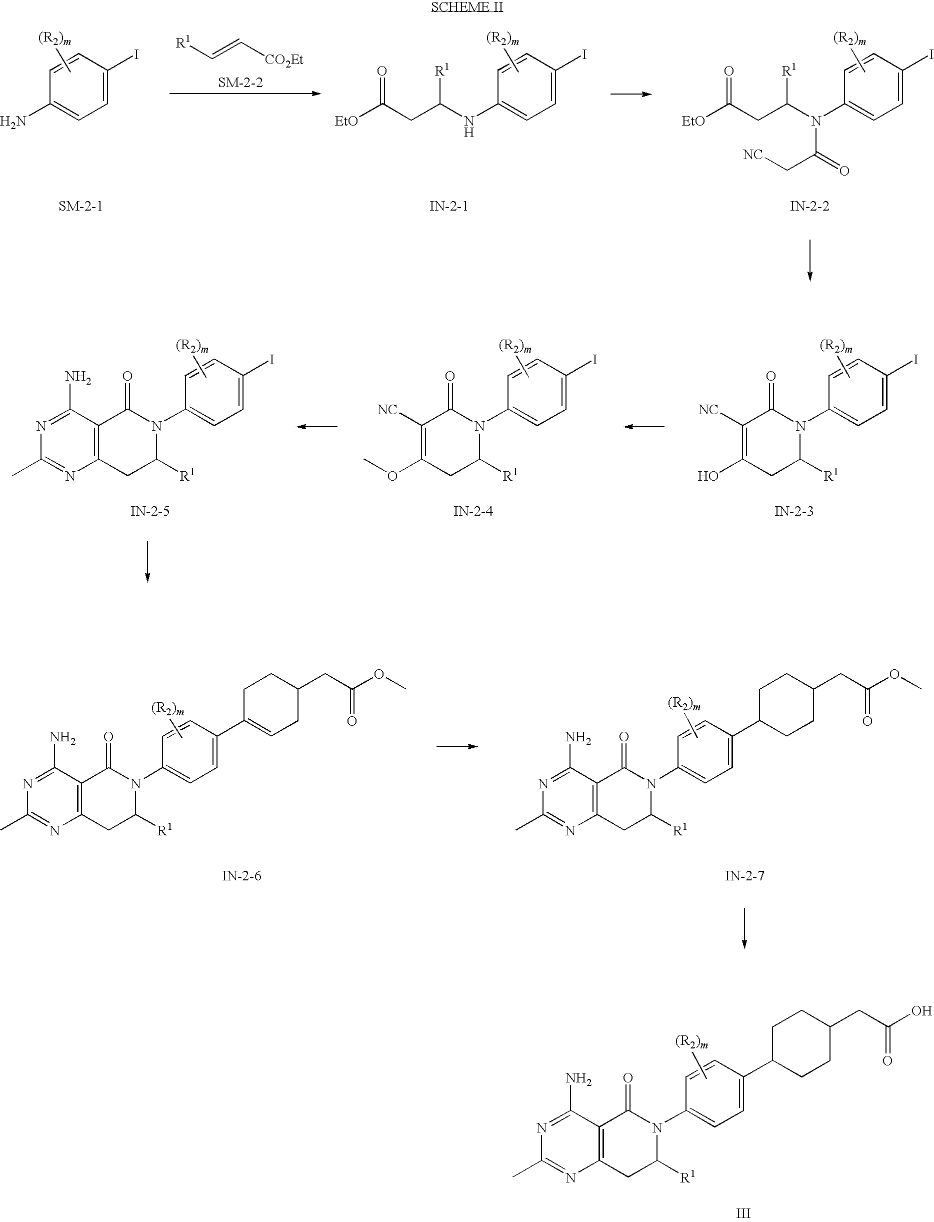

The desired starting materials (SM-2-1) such as 4-iodobenzenamine, i.e. where R² is H, and (SM-2-2) such as ethyl acrylate, i.e. where R¹ is H, and ethyl crotonate, i.e. where R¹ is methyl, may be purchased from commercial sources. Substituted 4-iodobenzenamines, i.e. where R² is, for example, fluorine, chlorine, or methyl, may also be purchased from commercial sources or prepared using methods well known in the art.

The two starting materials can be coupled together in the presence of acid, e.g. acetic acid, to form intermediate (IN-2-1). Cyanoacetic acid is then added to the secondary amino group of intermediate (IN-2-1) via an amide coupling using procedures well known to those of skill in the art, e.g., addition of cyanoacetic acid in the presence of an activator such as N—N′-diisopropylcarbodiimide (DIC) or 2-(7-aza-1H-benzotriazole-1-yl)-1,1,3,3-tetramethyluronium hexafluorophosphate (HATU) and a mild base, such as 4-dimethylaminopyridine (DMAP), in an appropriate solvent such as acetonitrile (ACN) to form the corresponding cyanoamide intermediate (IN-2-2). Formation of the lactam (IN-2-3) can be achieved by treatment with a base such as sodium ethoxide (EtONa) in an appropriate solvent, such as ethanol (EtOH). Methylation via the addition of trimethylsilyl-diazomethane (TMS-diazomethane) provides the methoxy lactam intermediate (IN-2-4). Addition of acetamidine to the methoxy lactam intermediate affords the corresponding aminopyrimidine (IN-2-5). Methyl 2-(cyclohex-3-enyl)acetate and the aminopyrimidine intermediate (IN-2-5) may be coupled together using a commercially available boron reagent, e.g. methyl 2-(4-(4,4,5,5-tetramethyl-1,3,2-dioxaborolan-2-yl)cyclohex-3-enyl)acetate, using procedures well known to those of skill in the art. The cross coupling reaction may occur in the presence of a palladium catalyst, e.g., tetrakis(triphenylphosphine)palladium (Pd(PPh₃)₄), and a weak base, e.g., cesium carbonate (Cs₂CO₃), in an appropriate solvent or mixture of solvents, e.g. tetrahydrofuran (THF), to afford intermediate (IN-2-6). The olefin is reduced via hydrogenation, e.g. treatment with hydrogen gas (H₂) in the presence of a catalyst such as palladium hydroxide on carbon (Pd(OH)₂ on carbon) in an appropriate solvent such as methanol. The resulting cyclohexane intermediate (IN-2-7) is a mixture of cis- and trans isomers which may be separated by chromatography using procedures well known to those of skill in the art. Formation of the corresponding carboxylic acid, a compound of Formula III, is accomplished by treatment with a strong base, such as potassium hydroxide (KOH) or sodium hydroxide (NaOH), in the presence of water and one or more polar solvents, e.g., methanol (MeOH) and tetrahydrofuran (THF).

Scheme IIa outlines an alternate procedure that may be used to prepare compounds of Formulas II and III.

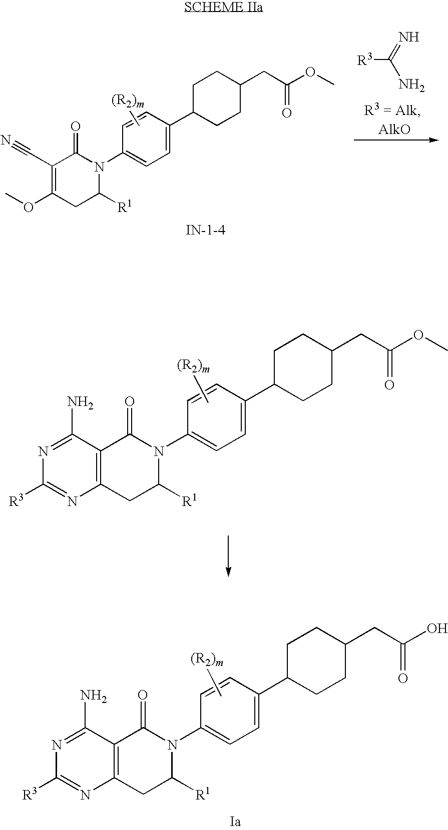

In Scheme IIa, treatment of the methyoxy lactam intermediate (IN-1-4) with the desired amidine in the presence of base, e.g. diisopropylethyl amine (DIPEA) in an appropriate solvent, e.g. methanol, affords the corresponding aminopyrimidine, which may be converted to a compound of Formula II or III using the methods described above.

Scheme III outlines the general procedure used to prepare compounds of Formula IV.

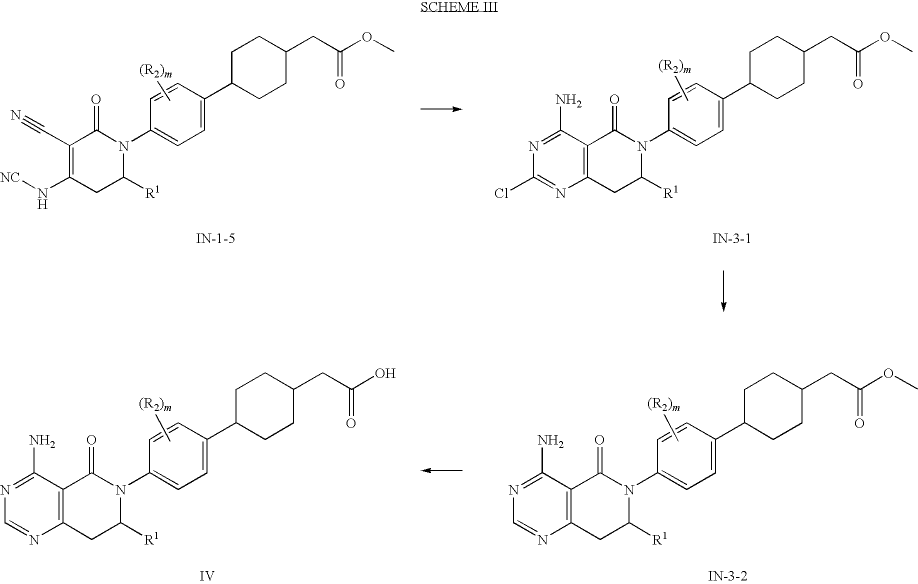

Compounds of Formula IV may be generally derived from intermediate compounds (IN-1-5). Treatment of the aminonitrile intermediate with HCl in dioxane at elevated temperatures affords the corresponding chloro-amino-pyrimidine intermediate (IN-3-1). Treatment of the chloro-amino-pyrimidine intermediate with H₂ gas in the presence of Pd(OH)₂ on carbon in an appropriate solvent, e.g. ethyl acetate (EtOAc), followed by the addition of an oxidizing agent, e.g. 2,3-dichloro-5,6-dicyanobenzoquinone (DDQ), in the appropriate solvent or mixture of solvents, e.g. dichloromethane (DCM) and acetonitrile (ACN), yields the dehalogenated aminopyrimidine (IN-3-2). Treatment of the aminopyrimidine with a strong base, e.g. potassium hydroxide (KOH), with water and one or more solvents, e.g. THF and MeOH, afforded compounds of Formula IV.

The compounds of the invention may contain asymmetric or chiral centers, and, therefore, exist in different stereoisomeric forms. Unless specified otherwise, it is intended that all stereoisomeric forms of the compounds of the invention as well as mixtures thereof, including racemic mixtures, form part of the invention. In addition, the invention embraces all geometric and positional isomers. For example, if a compound of the invention incorporates a double bond or a fused ring, both the cis- and trans-forms, as well as mixtures, are embraced within the scope of the invention.

Diastereomeric mixtures can be separated into their individual diastereoisomers on the basis of their physical chemical differences by methods well known to those skilled in the art, such as by chromatography and/or fractional crystallization. Enantiomers can be separated by converting the enantiomeric mixture into a diastereomeric mixture by reaction with an appropriate optically active compound (e.g., chiral auxiliary such as a chiral alcohol or Mosher's acid chloride), separating the diastereoisomers and converting (e.g., hydrolyzing) the individual diastereoisomers to the corresponding pure enantiomers. Also, some of the compounds of the invention may be atropisomers (e.g., substituted biaryls) and are considered as part of this invention. Enantiomers can also be separated by use of a chiral HPLC column. Alternatively, the specific stereoisomers may be synthesized by using an optically active starting material, by asymmetric synthesis using optically active reagents, substrates, catalysts or solvents, or by converting one stereoisomer into the other by asymmetric transformation.

It is also possible that the intermediates and compounds of the invention may exist in different tautomeric forms, and all such forms are embraced within the scope of the invention. The term “tautomer” or “tautomeric form” refers to structural isomers of different energies which are interconvertible via a low energy barrier. For example, proton tautomers (also known as prototropic tautomers) include interconversions via migration of a proton, such as keto-enol and imine-enamine isomerizations. A specific example of a proton tautomer is the imidazole moiety where the proton may migrate between the two ring nitrogens. Valence tautomers include interconversions by reorganization of some of the bonding electrons.

Certain compounds of the invention may exist in different stable conformational forms which may be separable. Torsional asymmetry due to restricted rotation about an asymmetric single bond, for example, because of steric hindrance or ring strain, may permit separation of different conformers.

The invention also embraces isotopically-labeled compounds of the invention which are identical to those recited herein, but for the fact that one or more atoms are replaced by an atom having an atomic mass or mass number different from the atomic mass or mass number usually found in nature. Examples of isotopes that can be incorporated into compounds of the invention include isotopes of hydrogen, carbon, nitrogen, oxygen, phosphorus, sulfur, fluorine, iodine, and chlorine, such as ²H, ³H, ¹¹C, ¹³C, ¹⁴C_{,} ¹³N, ¹⁵N, ¹⁵O, ¹⁷O, ¹⁸O, ³¹P, ³²P_{,} ³⁵S_{,} ¹⁸F, ¹²³I, ¹²⁵I and ³⁶Cl, respectively.

Certain isotopically-labeled compounds of the invention (e.g., those labeled with ³H and ¹⁴C) are useful in compound and/or substrate tissue distribution assays. Tritiated (i.e., ³H) and carbon-14 (i.e., ¹⁴C) isotopes may be used for their ease of preparation and detectability. Further, substitution with heavier isotopes such as deuterium (i.e., ²H) may afford certain therapeutic advantages resulting from greater metabolic stability (e.g., increased in vivo half-life or reduced dosage requirements) and hence may be used in some circumstances. Positron emitting isotopes such as ¹⁶O, ¹³N, ¹¹C, and ¹⁸F are useful for positron emission tomography (PET) studies to examine substrate occupancy. Isotopically labeled compounds of the invention can generally be prepared by following procedures analogous to those disclosed in the Schemes and/or in the Examples herein below, by substituting an isotopically labeled reagent for a non-isotopically labeled reagent.

Certain compounds of the invention may exist in more than one crystal form (generally referred to as “polymorphs”). Polymorphs may be prepared by crystallization under various conditions, for example, using different solvents or different solvent mixtures for recrystallization; crystallization at different temperatures; and/or various modes of cooling, ranging from very fast to very slow cooling during crystallization. Polymorphs may also be obtained by heating or melting the compound of the invention followed by gradual or fast cooling. The presence of polymorphs may be determined by solid probe NMR spectroscopy, IR spectroscopy, differential scanning calorimetry, powder X-ray diffraction or such other techniques.

Compounds of the invention are useful for treating diseases, conditions and/or disorders modulated by the inhibition of the DGAT-1 enzyme; therefore, another embodiment of the invention is a pharmaceutical composition comprising a therapeutically effective amount of a compound of the invention and a pharmaceutically acceptable excipient, diluent or carrier. The compounds of the invention (including the compositions and processes used therein) may also be used in the manufacture of a medicament for the therapeutic applications described herein.

A typical formulation is prepared by mixing a compound of the invention and a carrier, diluent or excipient. Suitable carriers, diluents and excipients are well known to those skilled in the art and include materials such as carbohydrates, waxes, water soluble and/or swellable polymers, hydrophilic or hydrophobic materials, gelatin, oils, solvents, water, and the like. The particular carrier, diluent or excipient used will depend upon the means and purpose for which the compound of the invention is being applied. Solvents are generally selected based on solvents recognized by persons skilled in the art as safe (GRAS) to be administered to a mammal. In general, safe solvents are non-toxic aqueous solvents such as water and other non-toxic solvents that are soluble or miscible in water. Suitable aqueous solvents include water, ethanol, propylene glycol, polyethylene glycols (e.g., PEG400, PEG300), etc. and mixtures thereof. The formulations may also include one or more buffers, stabilizing agents, surfactants, wetting agents, lubricating agents, emulsifiers, suspending agents, preservatives, antioxidants, opaquing agents, glidants, processing aids, colorants, sweeteners, perfuming agents, flavoring agents and other known additives to provide an elegant presentation of the drug (i.e., a compound of the invention or pharmaceutical composition thereof) or aid in the manufacturing of the pharmaceutical product (i.e., medicament).

The formulations may be prepared using conventional dissolution and mixing procedures. For example, the bulk drug substance (i.e., compound of the invention or stabilized form of the compound (e.g., complex with a cyclodextrin derivative or other known complexation agent)) is dissolved in a suitable solvent in the presence of one or more of the excipients described above. The compound of the invention is typically formulated into pharmaceutical dosage forms to provide an easily controllable dosage of the drug and to give the patient an elegant and easily handleable product.

The pharmaceutical composition (or formulation) for application may be packaged in a variety of ways depending upon the method used for administering the drug. Generally, an article for distribution includes a container having deposited therein the pharmaceutical formulation in an appropriate form. Suitable containers are well-known to those skilled in the art and include materials such as bottles (plastic and glass), sachets, ampoules, plastic bags, metal cylinders, and the like. The container may also include a tamper-proof assemblage to prevent indiscreet access to the contents of the package. In addition, the container has deposited thereon a label that describes the contents of the container. The label may also include appropriate warnings.

The invention further provides a method of treating diseases, conditions and/or disorders modulated by the inhibition of the DGAT-1 enzyme in an animal that includes administering to an animal in need of such treatment a therapeutically effective amount of a compound of the invention or a pharmaceutical composition comprising an effective amount of a compound of the invention and a pharmaceutically acceptable excipient, diluent, or carrier. The method is particularly useful for treating diseases, conditions and/or disorders that benefit from the inhibition of DGAT-1.

One aspect of the invention is the treatment of obesity, and obesity-related disorders (e.g., overweight, weight gain, or weight maintenance).

Obesity and overweight are generally defined by body mass index (BMI), which is correlated with total body fat and estimates the relative risk of disease. BMI is calculated by weight in kilograms divided by height in meters squared (kg/m²). Overweight is typically defined as a BMI of 25-29.9 kg/m², and obesity is typically defined as a BMI of 30 kg/m². See, e.g., National Heart, Lung, and Blood Institute, Clinical Guidelines on the Identification, Evaluation, and Treatment of Overweight and Obesity in Adults, The Evidence Report, Washington, D.C.: U.S. Department of Health and Human Services, NIH publication no. 98-4083 (1998).

Another aspect of the invention is for the treatment or delaying the progression or onset of diabetes or diabetes-related disorders including Type 1 (insulin-dependent diabetes mellitus, also referred to as “IDDM”) and Type 2 (noninsulin-dependent diabetes mellitus, also referred to as “NIDDM”) diabetes, impaired glucose tolerance, insulin resistance, hyperglycemia, and diabetic complications (such as atherosclerosis, coronary heart disease, stroke, peripheral vascular disease, nephropathy, hypertension, neuropathy, and retinopathy).

Yet another aspect of the invention is the treatment of diabetes- or obesity-related co-morbidities, such as metabolic syndrome. Metabolic syndrome includes diseases, conditions or disorders such as dyslipidemia, hypertension, insulin resistance, diabetes (e.g., Type 2 diabetes), weight gain, coronary artery disease and heart failure. For more detailed information on Metabolic Syndrome, see, e.g., Zimmet, P. Z., et al., “The Metabolic Syndrome Perhaps an Etiologic Mystery but Far From a Myth—Where Does the International Diabetes Federation Stand?,” Diabetes & Endocrinology, 7(2), (2005); and Alberti, K. G., et al., “The Metabolic Syndrome—A New Worldwide Definition,” Lancet, 366, 1059-62 (2005). Administration of the compounds of the invention may provide a statistically significant (p<0.05) reduction in at least one cardiovascular disease risk factpr, such as lowering of plasma leptin, C-reactive protein (CRP) and/or cholesterol, as compared to a vehicle control containing no drug. The administration of compounds of the invention may also provide a statistically significant (p<0.05) reduction in glucose serum levels.

In yet another aspect of the invention, the condition treated is impaired glucose tolerance, hyperglycemia, diabetic complications such as sugar cataracts, diabetic neuropathy, diabetic nephropathy, diabetic retinopathy and diabetic cardiomyopathy, anorexia nervosa, bulimia, cachexia, hyperuricemia, hyperinsulinemia, hypercholesterolemia, hyperlipidemia, dyslipidemia, mixed dyslipidemia, hypertriglyceridemia, nonalcoholic fatty liver disease, atherosclerosis, arteriosclerosis, acute heart failure, congestive heart failure, coronary artery disease, cardiomyopathy, myocardial infarction, angina pectoris, hypertension, hypotension, stroke, ischemia, ischemic reperfusion injury, aneurysm, restenosis, vascular stenosis, solid tumors, skin cancer, melanoma, lymphoma, breast cancer, lung cancer, colorectal cancer, stomach cancer, esophageal cancer, pancreatic cancer, prostate cancer, kidney cancer, liver cancer, bladder cancer, cervical cancer, uterine cancer, testicular cancer and ovarian cancer.

The invention also relates to therapeutic methods for treating the above described conditions in a mammal, including a human, wherein a compound of this invention is administered as part of an appropriate dosage regimen designed to obtain the benefits of the therapy. The appropriate dosage regimen, the amount of each dose administered and the intervals between doses of the compound will depend upon the compound of this invention being used, the type of pharmaceutical compositions being used, the characteristics of the subject being treated and the severity of the conditions.

The invention also provides pharmaceutical compositions which comprise a therapeutically effective amount of a compound, or a pharmaceutically acceptable salt thereof, in admixture with at least one pharmaceutically acceptable excipient. The compositions include those in a form adapted for oral, topical or parenteral use and can be used for the treatment of diabetes and related conditions as described above.

The composition can be formulated for administration by any route known in the art, such as subdermal, inhalation, oral, topical, parenteral, etc. The compositions may be in any form known in the art, including but not limited to tablets, capsules, powders, granules, lozenges, or liquid preparations, such as oral or sterile parenteral solutions or suspensions.

Tablets and capsules for oral administration may be in unit dose presentation form, and may contain conventional excipients such as binding agents, for example syrup, acacia, gelatin, sorbitol, tragacanth, or polyvinylpyrollidone; fillers, for example lactose, sugar, maize-starch, calcium phosphate, sorbitol or glycine; tabletting lubricants, for example magnesium stearate, talc, polyethylene glycol or silica; disintegrants, for example potato starch; or acceptable wetting agents such as sodium lauryl sulphate. The tablets may be coated according to methods well known in normal pharmaceutical practice.

Oral liquid preparations may be in the form of, for example, aqueous or oily suspensions, solutions, emulsions, syrups or elixirs, or may be presented as a dry product for reconstitution with water or other suitable vehicle before use. Such liquid preparations may contain conventional additives, such as suspending agents, for example sorbitol, methyl cellulose, glucose syrup, gelatin, hydroxyethyl cellulose, carboxymethyl cellulose, aluminium stearate gel or hydrogenated edible fats, emulsifying agents, for example lecithin, sorbitan monooleate, or acacia; non-aqueous vehicles (which may include edible oils), for example almond oil, oily esters such as glycerin, propylene glycol, or ethyl alcohol; preservatives, for example methyl or propyl p-hydroxybenzoate or sorbic acid, and, if desired, conventional flavoring or coloring agents.

For parenteral administration, fluid unit dosage forms are prepared utilizing the compound and a sterile vehicle, water being preferred. The compound, depending on the vehicle and concentration used, can be either suspended or dissolved in the vehicle or other suitable solvent. In preparing solutions, the compound can be dissolved in water for injection and filter sterilized before filling into a suitable vial or ampoule and sealing. Advantageously, agents such as local anesthetics, preservatives and buffering agents etc. can be dissolved in the vehicle. To enhance the stability, the composition can be frozen after filling into the vial and the water removed under vacuum. The dry lyophilized powder is then sealed in the vial and an accompanying vial of water for injection may be supplied to reconstitute the liquid prior to use. Parenteral suspensions are prepared in substantially the same manner except that the compound is suspended in the vehicle instead of being dissolved and sterilization cannot be accomplished by filtration. The compound can be sterilized by exposure to ethylene oxide before suspending in the sterile vehicle. Advantageously, a surfactant or wetting agent is included in the composition to facilitate uniform distribution of the compound.

The compositions may contain, for example, from about 0.1% to about 99 by weight, of the active material, depending on the method of administration. Where the compositions comprise dosage units, each unit will contain, for example, from about 0.1 to 900 mg of the active ingredient, more typically from 1 mg to 250 mg, or 0.01 mg/kg/day to 30 mg/kg/day, such as 0.01 mg/kg/day to 5 mg/kg/day of active compound in single or divided doses.

Compounds of the invention can be formulated for administration in any convenient way for use in human or veterinary medicine, by analogy with other anti-diabetic agents. Such methods are known in the art and have been summarized above. For a more detailed discussion regarding the preparation of such formulations; the reader's attention is directed to Remington's Pharmaceutical Sciences, 21^{st} Edition, by University of the Sciences in Philadelphia.

It is also noted that the compounds of the invention can be used in sustained release, controlled release, and delayed release formulations, which forms are also well known to one of ordinary skill in the art.

The compounds of this invention may also be used in conjunction with other pharmaceutical agents for the treatment of the diseases, conditions and/or disorders described herein. Therefore, methods of treatment that include administering compounds of the invention in combination with other pharmaceutical agents are also provided. Suitable pharmaceutical agents that may be used in combination with the compounds of the invention include anti-obesity agents (including appetite suppressants), anti-diabetic agents, anti-hyperglycemic agents, lipid lowering agents, and anti-hypertensive agents.

Suitable anti-diabetic agents include an acetyl-CoA carboxylase-2 (ACC-2) inhibitor, a phosphodiesterase (PDE)-10 inhibitor, a sulfonylurea (e.g., acetohexamide, chlorpropamide, diabinese, glibenclamide, glipizide, glyburide, glimepiride, gliclazide, glipentide, gliquidone, glisolamide, tolazamide, and tolbutamide), a meglitinide, an α-amylase inhibitor (e.g., tendamistat, trestatin and AL-3688), an α-glucoside hydrolase inhibitor (e.g., acarbose), an α-glucosidase inhibitor (e.g., adiposine, camiglibose, emiglitate, miglitol, voglibose, pradimicin-Q, and salbostatin), a PPARy agonist (e.g., balaglitazone, ciglitazone, darglitazone, englitazone, isaglitazone, pioglitazone, rosiglitazone and troglitazone), a PPAR α/γ agonist (e.g., CLX-0940, GW-1536, GW-1929, GW-2433, KRP-297, L-796449, LR-90, MK-0767 and SB-219994), a biguanide (e.g., metformin), a glucagon-like peptide 1 (GLP-1) agonist (e.g., exendin-3 and exendin-4), a protein tyrosine phosphatase-1B (PTP-1B) inhibitor (e.g., trodusquemine, hyrtiosal extract, and compounds disclosed by Zhang, S., et al., Drug Discovery Today, 12(9/10), 373-381 (2007)), SIRT-1 inhibitor (e.g., reservatrol), a dipeptidyl peptidease IV (DPP-IV) inhibitor (e.g., sitagliptin, vildagliptin, alogliptin and saxagliptin), an insulin secreatagogue, a fatty acid oxidation inhibitor, an A2 antagonist, a c-jun amino-terminal kinase (JNK) inhibitor, insulin, an insulin mimetic, a glycogen phosphorylase inhibitor, a VPAC2 receptor agonist and a glucokinase activator. Exemplary anti-diabetic agents are metformin and DPP-IV inhibitors (e.g., sitagliptin, vildagliptin, alogliptin and saxagliptin).

Suitable anti-obesity agents include 11β-hydroxy steroid dehydrogenase-1 (11β-HSD type 1) inhibitors, stearoyl-CoA desaturase-1 (SCD-1) inhibitor, MCR-4 agonists, cholecystokinin-A (CCK A)agonists, monoamine reuptake inhibitors (such as sibutramine), sympathomimetic agents, β₃ adrenergic agonists, dopamine agonists (such as bromocriptine), melanocyte-stimulating hormone analogs, 5HT2c agonists, melanin concentrating hormone antagonists, leptin (the OB protein), leptin analogs, leptin agonists, galanin antagonists, lipase inhibitors (such as tetrahydrolipstatin, i.e. orlistat), anorectic agents (such as a bombesin agonist), neuropeptide-γ antagonists (e.g., NPY Y5 antagonists), PYY₃₋₃₆ (including analogs thereof), thyromimetic agents, dehydroepiandrosterone or an analog thereof, glucocorticoid agonists or antagonists, orexin antagonists, glucagon-like peptide-1 agonists, ciliary neurotrophic factors (such as Axokine™ available from Regeneron Pharmaceuticals, Inc., Tarrytown, N.Y. and Procter & Gamble Company, Cincinnati, Ohio), human agouti-related protein (AGRP) inhibitors, ghrelin antagonists, histamine 3 antagonists or inverse agonists, neuromedin U agonists, MTP/ApoB inhibitors (e.g., gut-selective MTP inhibitors, such as dirlotapide), opioid antagonist, orexin antagonist, and the like.

Exemplary anti-obesity agents for use in the combination aspects of the invention include gut-selective MTP inhibitors (e.g., dirlotapide, mitratapide and implitapide, R56918 (CAS No. 403987) and CAS No. 913541-47-6), CCKa agonists (e.g., N-benzyl-2-[4-(1H-indol-3-ylmethyl)-5-oxo-1-phenyl-4,5-dihydro-2,3,6,10b-tetraaza-benzo[e]azulen-6-yl]-N-isopropyl-acetamide described in PCT Publication No. WO 2005/116034 or US Publication No. 2005-0267100 A1), 5HT2c agonists (e.g., lorcaserin), MCR4 agonist (e.g., compounds described in U.S. Pat. No. 6,818,658), lipase inhibitor (e.g., Cetilistat), PYY₃₋₃₆(as used herein “PYY₃₋₃₆” includes analogs, such as peglated PYY₃₋₃₆ e.g., those described in US Publication 2006/0178501), opioid antagonists (e.g., naltrexone), oleoyl-estrone (CAS No. 180003-17-2), obinepitide (TM30338), pramlintide (Symlin®), tesofensine (NS2330), leptin, liraglutide, bromocriptine, orlistat, exenatide (Byetta®), AOD-9604 (CAS No. 221231-10-3) and sibutramine. Compounds of the invention and combination therapies may be administered in conjunction with exercise and a sensible diet.

Embodiments of the invention are illustrated by the following Examples. It is to be understood, however, that the embodiments of the invention are not limited to the specific details of these Examples, as other variations thereof will be known, or apparent in light of the instant disclosure, to one of ordinary skill in the art.

### EXAMPLES

Unless specified otherwise, starting materials are generally available from commercial sources such as Aldrich Chemicals Co. (Milwaukee, Wis.), Lancaster Synthesis, Inc. (Windham, N.H.), Acros Organics (Fairlawn, N.J.), Maybridge Chemical Company, Ltd. (Cornwall, England), Tyger Scientific (Princeton, N.J.), and AstraZeneca Pharmaceuticals (London, England).

### General Experimental Procedures

NMR spectra were recorded on a Varian Unity™ 400 (available from Varian Inc., Palo Alto, Calif.) at room temperature at 400 MHz for proton. Chemical shifts are expressed in parts per million (δ) relative to residual solvent as an internal reference. The peak shapes are denoted as follows: s, singlet; d, doublet; dd, doublet of doublet; t, triplet; q, quartet; m, multiplet; bs, broad singlet; 2s, two singlets. Atmospheric pressure chemical ionization mass spectra (APCI) were obtained on a Fisons' Platform II Spectrometer (carrier gas: acetonitrile: available from Micromass Ltd, Manchester, UK). Chemical ionization mass spectra (CI) were obtained on a Hewlett-Packard™ 5989 instrument (ammonia ionization, PBMS: available from Hewlett-Packard Company, Palo Alto, Calif.). Electrospray ionization mass spectra (ES) were obtained on a Waters™ ZMD instrument (carrier gas: acetonitrile: available from Waters Corp., Milford, Mass.). High resolution mass spectra (HRMS) were obtained on an Agilent™ Model 6210 using time of flight method. Where the intensity of chlorine or bromine-containing ions are described, the expected intensity ratio was observed (approximately 3:1 for ³⁵Cl/³⁷Cl containing ions and 1:1 for ⁷⁹Br/⁸¹Br-containing ions) and the intensity of only the lower mass ion is given. In some cases only representative ¹H NMR peaks are given. Optical rotations were determined on a PerkinElmer™ 241 polarimeter (available from PerkinElmer Inc., Wellesley, Mass.) using the sodium D line (λ=589 nm) at the indicated temperature and are reported as follows [α]_{D}^{temp}, concentration (c=g/100 ml), and solvent.

Column chromatography was performed with either Baker silica gel (40 μm; J. T. Baker, Phillipsburg, N.J.) or Silica Gel 50 (EM Sciences™, Gibbstown, N.J.) in glass columns or in Flash 40 Biotage™ columns (ISC, Inc., Shelton, Conn.) or Biotage™ SNAP cartridge KPsil or Redisep Rf silica (from Teledyne™ Isco™) under low nitrogen pressure.

### Starting Materials

Methyl [trans-4-[4-[[(trifluoromethyl)sulfonyl]oxy]phenyl]cyclohexyl]acetate was prepared as described for Compound 56 in US Application No. 2004/0209871.

### Preparation of Key Intermediates

### Preparation of Ethyl N-{4-[trans-4-(2-methoxy-2-oxoethyl)cyclohexyl]phenyl}-beta-alaninate

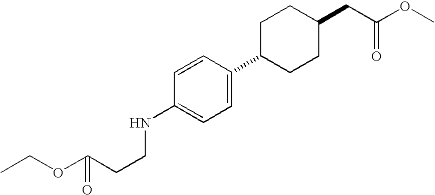

Methyl [trans-4-[4-[[(trifluoromethyl)sulfonyl]oxy]phenyl]cyclohexyl]acetate (13.0 g, 34.1 mmol), ethyl 3-aminopropanoate (6.3 g, 41 mmol), and cesium carbonate (27.8 g, 85.4 mmol) were all combined in a reaction vessel and suspended in toluene (230 mL). Nitrogen gas was bubbled through the stirred mixture for 20 minutes. Palladium (II) acetate (614 mg, 2.73 mmol), 2-dicyclohexylphosphino-2′, 4′, 6′-tri-1-propyl-1,1-biphenyl (1.3 g, 2.73 mmol) and DIPEA (7.74 mL, 44.4 mmol) were added to the reaction and nitrogen was bubbled through the mixture for 5 more minutes. The vessel was capped and the mixture was stirred at +120° C. for 20 hours. The reaction was cooled to room temperature and concentrated. Purification was done by chromatography (silica gel column with 40% EtOAc:heptane) to give 6.75 g of ethyl N-{4-[trans-4-(2-methoxy-2-oxoethyl)cyclohexyl]phenyl}-beta-alaninate (57% yield).

¹H NMR (500 MHz, CDCl₃): δ ppm 1.09-1.19 (m, 2H) 1.27 (t, 3H) 1.40-1.53 (m, 2H) 1.81-1.93 (m, 6H) 2.26 (d, 2H) 2.62 (t, 2H) 3.44 (t, 2H) 3.69 (s, 3H) 4.16 (q, 2H) 6.60 (d, 2H) 7.04 (d, 2H). m/z=348.5 (M+1).

### Preparation of ethyl N-(cyanoacetyl)-N-[4-{trans-4-(2-methoxy-2 oxoethyl)cyclohexyl]phenyl}-beta-alaninate

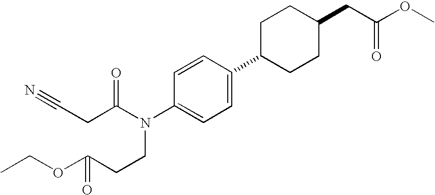

To a stirred mixture of ethyl N-{4-[trans-4-(2-methoxy-2-oxoethyl)cyclohexyl]phenyl}-beta-alaninate (6.75 g, 19.43 mmol), cyanoacetic acid (1.74 g, 20.4 mmol) and DMF (100 mL) was added diisopropylcarbodiimide (3.16 mL, 20.4 mmol) dropwise at 0° C. The reaction was allowed to warm up and stir at room temperature for 3 days. EtOAc (200 mL) and heptane (400 mL) were added to the mixture which was then washed with water (3×300 mL). The organic phase was dried over magnesium sulfate and loaded on silica gel. The column was eluted with a gradient from 30-90% EtOAc:heptane to give ethyl N-(cyanoacetyl)-N-{4-[trans-4-(2-methoxy-2-oxoethyl)cyclohexyl]phenyl}-beta-alaninate (5.2 g, 64% yield).

¹H NMR (500 MHz, CDCl₃): δ ppm 1.14-1.21 (m, 2H), 1.21 (t, 3H), 1.48-1.56 (m, 2H) 1.84-1.89 (m, 1H), 1.90-1.96 (m, 4H), 2.28 (d, 2H), 2.50-2.57 (m, 1H), 2.59 (t, 2H), 3.19 (s, 2H), 3.71 (s, 3H), 4.02 (t, 2H), 4.08 (q, 2H), 7.14 (d, 2H), 7.30 (d, 2H). m/z=415.5 (M+1).

### Preparation of methyl 2-{trans-4-(4-(3-cyano-4-hydroxy-2-oxo-5,6-dihydropyridin-1(2H)-yl)phenyl)cyclohexyl}acetate

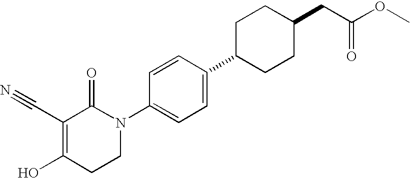

A mixture of ethyl N-(cyanoacetyl)-N-{4-[trans-4-(2-methoxy-2-oxoethyl)cyclohexyl]phenyl}-beta-alaninate (7.3 g, 18 mmol) and 1,5-diazabicyclo(5.4.0)undec-7-ene (3.16 mL, 21.1 mmol) was diluted in MeOH (138 mL) and heated at +80° C. for 3 hours. The reaction was concentrated and then diluted with EtOAc (70 mL), heptane (70 mL), 1M HCl (42 mL) and water (240 mL). The mixture was stirred at room temperature for 2 hours. The resulting precipitate was filtered off and dried in a vacuum oven at +40° C. overnight to give methyl {trans-4-(4-(3-cyano-4-hydroxy-2-oxo-5,6-dihydropyridin-1(2H)-yl)phenyl)cyclohexyl)}acetate as an off-white solid (5.2 g, 80% yield).

¹H NMR (500 MHz, DMSO-d₆): 6 ppm 1.08-1.19 (m, 2H), 1.40-1.51 (m, 2H), 1.72-1.83 (m, 6H), 2.24 (d, 2H), 2.44 (t, 1H), 2.80 (t, 2H), 3.60 (s, 3H), 3.77 (t, 2H), 7.15 (d, 2H), 7.22 (d, 2H). m/z=367.4 (M−1).

### Preparation of methyl {trans-4-[4-(5-cyano-4-methoxy-6-oxo-3,6-dihydropyridin-1(2H)-yl)phenyl]cyclohexyl}acetate

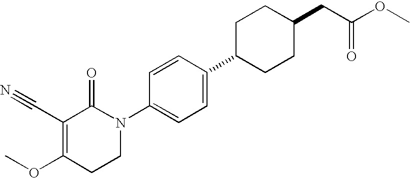

To a 5 L round-bottomed flask was added methyl {trans-4-(4-(3-cyano-4-hydroxy-2-oxo-5,6-dihydropyridin-1(2H)-yl)phenyl)cyclohexyl)}acetate (245 g, 665 mmol), DCM (3.22 L), and DMF (32.1 mL, 415 mmol). Oxalyl chloride (106.33 mL, 1.23 moles) was slowly added to the stirred solution. During addition the reaction was cooled to 15° C. and kept between 15-25° C. The reaction was stirred at room temperature until the reaction mixture was clear. DCM was removed and MeOH (3.92 L) was added. The reaction was heated to 65° C. for an additional 12 hours until all of the chlorine intermediate was gone. The solid was filtered to produce methyl 2-{trans-4-[4-(5-cyano-4-methoxy-6-oxo-3,6-dihydropyridin-1(2H)-yl)phenyl]cyclohexyl}acetate (230.0 g, 90.4% yield).

¹H NMR (400 MHz, DMSO-d₆) 8 ppm 1.03-1.18 (m, 2H) 1.36-1.51 (m, 2H) 1.76 (d, J=9.76 Hz, 5H) 2.21 (d, J=6.64 Hz, 2H) 2.37-2.45 (m, 1H) 3.01 (t, J=6.83 Hz, 2H) 3.56 (s, 3H) 3.79 (t, J=6.83 Hz, 2H) 4.00 (s, 3H) 7.11-7.24 (m, 4H). m/z=383.5 (M+1).

### Preparation of methyl (trans-4-{4-[5-cyano-4-(cyanoamino)-6-oxo-3,6-dihydropyridin-1(2H)-yl]phenyl}cyclohexyl)acetate

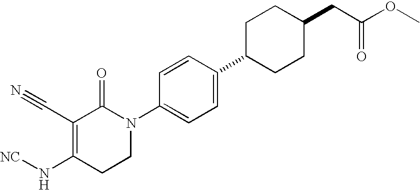

To a 5 L round-bottomed flask was added methyl {trans-4-[4-(5-cyano-4-methoxy-6-oxo-3,6-dihydropyridin-1(2H)-yl)phenyl]cyclohexyl}acetate (230 g, 601.33 mmol), cyanamide (55.62 g, 1.32 moles) and MeOH (2.76 L). Sodium methoxide (107.2 g, 1.98 moles) was slowly added. The reaction was stirred at room temperature for 1 hour. The mixture was cooled to 10° C. 1N HCl (1.6 L, to pH 3) and water (1.6 L) were added. MeOH was removed in vacuo. 1N HCL (0.4 L) was added. The slurry was stirred at room temperature for 15 minutes and filtered. The cake was washed with water (0.5 L) and dried to give methyl (trans-4-{4-[5-cyano-4-(cyanoamino)-6-oxo-3,6-dihydropyridin-1(2H)-yl]phenyl}cyclohexyl)acetate as an off-white powder (233.0 g, 98.7% yield). This material was used in the next reaction without characterization or purification.

### Preparation of methyl {trans-4-[4-(4-amino-2-methoxy-5-oxo-7,8-dihydropyrido[4,3-a]pyrimidin-6(5H)-yl)phenyl]cyclohexyl}acetate

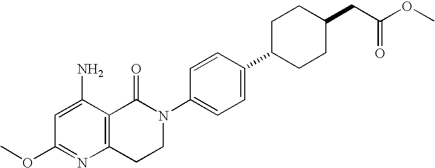

A mixture of methyl (trans-4-{4-[5-cyano-4-(cyanoamino)-6-oxo-3,6-dihydropyridin-1(2H)-yl]phenyl}cyclohexyl)acetate (11.5 g, 30.1 mmol), 2-methylisourea (6.65 g, 60.1 mmol) and DIPEA (21.0 mL, 120 mmol) in MeOH (400 mL) was warmed to +70° C. over 20 minures and stirred at +70° C. for one hour. The mixture was filtered and the resulting white solid was refluxed in MeOH (100 mL) and DCM (150 mL) for two hours. The mixture was filtered. The filtrate was concentrated and the residue was purified by chromatography (120 g silica gel column 1-5% MeOH:DCM) to give methyl {trans-4-[4-(4-amino-2-methoxy-5-oxo-7,8-dihydropyrido[4,3-a]pyrimidin-6(5H)-yl)phenyl]cyclohexyl}acetate as a white solid (4.3 g, 48% yield).

¹H NMR (400 MHz, DMSO-d₆): δ ppm 1.03-1.18 (m, 2H), 1.36-1.52 (m, 2H), 1.64-1.84 (m, 5H), 2.21 (d, J=6.83 Hz, 2H), 2.38-2.45 (m, 1H), 2.90 (t, J=6.83 Hz, 2H), 3.57 (s, 3H), 3.79-3.88 (m, 5H), 7.17-7.25 (m, 4H), 7.73 (d, J=3.90 Hz, 1H), 8.34 (d, J=4.10 Hz, 1H). m/z=425.5 (M+1).

### Preparation of N-(3-ethoxybut-3-enyl)-4-iodobenzenamine

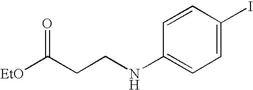

4-Iodoiniline (50 g, 227 mmol) and ethylacrylate (24 ml, 227 m mol) were dissolved in acetic acid (200 mL). The mixture was heated to 75° C. for 16 hours. The reaction mixture was then concentrated and the residue was diluted with water. It was extracted with ethyl acetate and the combined organic layers were washed with water and brine. It was dried over Na₂SO₄ and evaporated to dryness. The crude material was purified by column chromatography using hexane-EtOAc as eluting solvents to yield N-(3-ethoxybut-3-enyl)-4-iodobenzenamine (57 g, 79% yield).

¹H NMR (400 MHz, CDCl₃): δ ppm 7.24 (m, 2H), 6.69 (m, 2H), 4.13 (q, J=7.2 Hz, 2H), 3.64 (m, 2H), 2.57 (m, 2H), 1.22 (t, J=7.2 Hz, 3H).

### Preparation of Ethyl 3-(2-cyano-N-(4-iodophenyl)acetamido)propanoate)

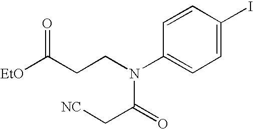

To a stirred solution of a mixture of N-(3-ethoxybut-3-enyl)-4-iodobenzenamine (20 g, 62 mmol) and cyano acetic acid (7.99 g, 94.04 mmol) in DMF (100 mL) was added diisoproprylcarbodiimide (8.6 g, 68 mmol) dropwise at 0° C. under N₂ atmosphere over 15 minutes. The resulting mixture was stirred for 1 hour at room temperature and diluted with 1:1 mixture of ethylacetate-hexane. The mixture was then filtered to remove the urea by-product. The filtrate was partitioned between dilute HCl (0.5 N) and ethylacetate. The organic phase was washed with brine, dried over sodium sulfate and concentrated. The crude product was purified by column chromatography using 230-400 mesh silica gel as a support and 20% ethylacetate in hexane as a eluting solvents to give ethyl 3-(2-cyano-N-(4-iodophenyl)acetamido)propanoate (17 g, 70% yield). It was re-purified over silica gel column using hexane containing 15% EtOAc. Compound obtained after column was dissolved in minimum ether and after cooling solid appeared which was filtered off to give 8 g (32% yield) of ethyl 3-(2-cyano-N-(4-iodophenyl)acetamido)propanoate.

¹H NMR (400 MHz, DMSO-d₆): δ ppm 7.80 (d, J=8.4 Hz, 2H), 6.98 (d, J=8.4 Hz, 2H), 4.04 (q, J=7.2 Hz, 2H), 3.96 (t, 2H), 3.18 (s, 2H), 2.53 (t, 2H), 1.21 (m, 3H).

### Preparation of 4-Hydroxy-1-(4-iodophenyl)-2-oxo-1,2,5,6-tetrahydropyridine-3-carbonitrile

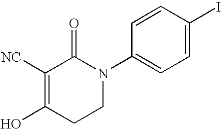

To a freshly prepared NaOEt (14.24 mmol) solution in ethanol was added a solution of ethyl 3-(2-cyano-N-(4-iodophenyl)acetamido)propanoate) (5 g, 12.95 ml/mol) dissolved in 60 mL dry ethanol dropwise at 0° C. The solution was then stirred at room temperature overnight. Ethanol was distilled off and the crude residue was dissolved in water. It was acidified with 2N HCl to pH-4. The precipitate was filtered and dried to give 4-hydroxy-1-(4-iodophenyl)-2-oxo-1,2,5,6-tetrahydropyridine-3-carbonitrile (2.9 g, 67% yield).

¹H NMR (400 MHz, DMSO-d₆): 5 ppm 7.69 (d, J=8.4 Hz, 2H), 7.08 (d, J=8.4 Hz, 2H), 3.75 (t, 2H), 2.79 (t, 2H).

### Preparation of 1-(4-Iodophenyl)-4-methoxy-2-oxo-1,2,5,6-tetrahydropyridine-3-carbonitrile

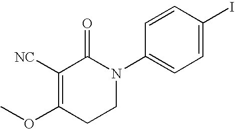

4-hydroxy-1-(4-iodophenyl)-2-oxo-1,2,5,6-tetrahydropyridine-3-carbonitrile (9 g, 26.4 mL mol) was taken up in 250 mL of carbinol. An excess ethereal solution of diazomethane (freshly prepared from 25 equivalents of nitrosomethylurea (NMU)) was added to the reaction mixture at 0° C. The reaction was stirred for 0.5 h at 0° C. and carbinol was removed. The crude product was washed thoroughly with water and dried to give 1-(4-iodophenyl)-4-methoxy-2-oxo-1,2,5,6-tetrahydropyridine-3-carbonitrile (2.9 g, 31% yield).

¹H NMR (400 MHz, DMSO-d₆): δ ppm 7.72 (d, J=8.4 Hz, 2H), 7.12 (d, J=8.4 Hz, 2H), 4.03 (s, 3H), 3.83 (t, 2H), 3.04 (t, 2H).

### Preparation of 4-Amino-6-(4-iodophenyl)-2-methyl-7,8-dihydropyrido[4,3-α]pyrimidin-5(6H)-one

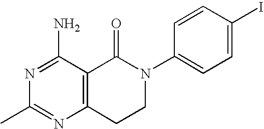

A mixture of 1-(4-iodophenyl)-4-methoxy-2-oxo-1,2,5,6-tetrahydropyridine-3-carbonitrile (40 mg, 0.10 mmol), acetamidine hydrochloride (87 mg, 0.801 mmol) and diisopropylethylamine (0.140 mL, 0.804 mmol) in MeOH (1.5 mL) was warmed from room temperature to +80° C. in 20 minutes and stirred at that temperature for 1 hour. The mixture was loaded on silica gel and eluted with a gradient from 70-100% EtOAc in heptane to give 4-amino-6-(4-iodophenyl)-2-methyl-7,8-dihydropyrido[4,3-d]pyrimidin-5(6H)-one (IN-2-5) as a colorless solid (26 mg, 59% yield).

¹H NMR (400 MHz, DMSO-d₆): 6 ppm 8.15 (s, 1H), 7.74 (m, 3H), 7.17 (d, J=8.4 Hz, 2H), 3.88 (m, 2H), 2.96 (m, 2H), 2.35 (s, 3H). m/z=381 (M+1).

### Preparation of Methyl 2-(4-(4-(4-amino-2-methyl-5-oxo-7,8-dihydropyrido[4,3-α]pyrimidin-6(5H)-yl)phenyl)cyclohex-3-enyl)acetate

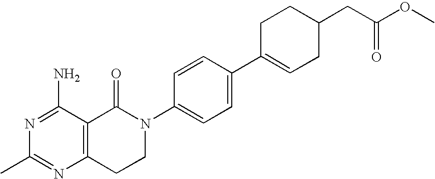

Palladium (0) tetrakis(triphenylphosphine) (13 mg, 0.011 mmol) and cesium carbonate (105 mg, 0.316 mmol) were added to a solution of 4-amino-6-(4-iodophenyl)-2-methyl-7,8-dihydropyrido[4,3-a]pyrimidin-5(6H)-one (100 mg, 0.263 mmol) and methyl 2-(4-(4,4,5,5-tetramethyl-1,3,2-dioxaborolan-2-yl)cyclohex-3-3nyl)acetate (73.7 mg, 0.263 mmol) in THF (2 mL) under N₂. The reaction mixture was refluxed with stirring for 16 hrs. The reaction mixture was then cooled, diluted with EtOAc, washed with water and brine, dried over MgSO₄ and concentrated. The crude material was purified by chromatography (12 g silica gel column 2-5% MeOH/DCM) to give methyl 2-(4-(4-(4-amino-2-methyl-5-oxo-7,8-dihydropyrido[4,3-a]pyrimidin-6(5H)-yl)phenyl)cyclohex-3-enyl)acetate as a yellow solid (82 mg, 77% yield).

¹H NMR (400 MHz, CHLOROFORM-d) δ ppm 1.36-1.51 (m, 1H) 1.75 (br. s., 1H) 1.82-1.98 (m, 2H) 2.14 (br. s., 1H) 2.32 (d, J=7.42 Hz, 2H) 2.37-2.53 (m, 5H) 3.06 (t, J=6.83 Hz, 2H) 3.67 (s, 3H) 3.94 (t, J=6.83 Hz, 2H) 5.70 (br. s., 1H) 5.97-6.11 (m, 1H) 7.18-7.26 (m, 2H) 7.40 (d, J=8.59 Hz, 2H) 8.52 (br. s., 1H) m/z=407.4 (M+1)

### Preparation of Methyl 2-(4-(4-(4-amino-2-methyl-5-oxo-7,8-dihydropyrido[4,3-α]pyrimidin-6(5H)-yl)phenyl)cyclohexy)acetate

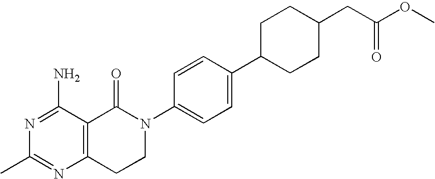

Methyl 2-(4-(4-(4-amino-2-methyl-5-oxo-7,8-dihydropyrido[4,3-a]pyrimidin-6(5H)-yl)phenyl)cyclohex-3-enyl)acetate (82 mg, 0.20 mmol) was dissolved in EtOH (10 mL) and EtOAc (10 mL). 20% Pd(OH)₂/C (40 mg) was added to the solution. The reaction mixture was shaken at 50 psi (3.4 atm) of H₂ for 16 hrs. The mixture was filtered through Celite to remove the catalyst and concentrated to give methyl 2-(4-(4-(4-amino-2-methyl-5-oxo-7,8-dihydropyrido[4,3-α]pyrimidin-6(5H)-yl)phenyl)cyclohexy)acetate as an off-white solid (80 mgs, 97% yield).

m/z=409.5 (M+1).

### Preparation of Methyl {trans-2-4-(4-(4-amino-2-chloro-5-oxo-7,8-dihydropyrido[4,3-α]pyrimidin-6(5H)-yl)phenyl}cyclohexyl)acetate

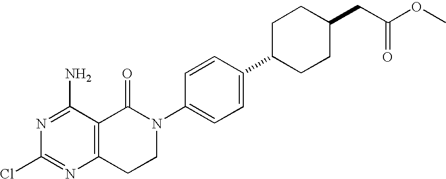

Cyanamide (115 mg, 2.74 mmol) was added to a stirred suspension of NaH (60% in mineral oil, 109 mg, 2.74 mmol) in dioxane (15 mL) at room temperature. After stirring for 15 minutes, methyl {trans-4-[4-(5-cyano-4-(cyanoamino)-6-oxo-3,6-dihydropyridin-1(2H)-yl)phenyl]cyclohexyl}acetate (700 mg, 1.83 mmol) was added and the mixture was stirred at room temperature for 4 hours. A solution of 4M HCl in dioxane (12.5 mL) was then added and the mixture was heated to 100° C. for 3 hours. After cooling, water (100 mL) was added and the mixture was extracted into EtOAc (2×50 mL). The combined organics were washed with brine (50 mL), dried (MgSO₄) and evaporated to afford the title compound, methyl {trans-2-4-(4-(4-amino-2-chloro-5-oxo-7,8-dihydropyrido[4,3-a]pyrimidin-6(5H)-yl)phenyl}cyclohexyl)acetate) (700 mg) which was used in the next reaction without characterization or purification.

### Preparation of Methyl 2-{trans-4-(4-(4-amino-5-oxo-7,7-dihydropyrido[4,3-α]pyrimidin-6(5H)-yl)phenyl}cyclohexyl)acetate

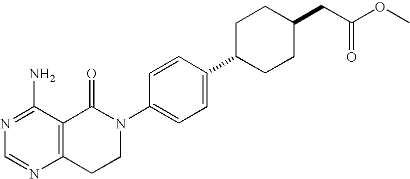

Methyl {trans-2-4-(4-(4-amino-2-chloro-5-oxo-7,8-dihydropyrido[4,3-a]pyrimidin-6(5H)-yl)phenyl}cyclohexyl)acetate) (700 mg) was dissolved in EtOAc (100 mL). To this was added palladium hydroxide (20% on carbon, 200 mg) and the mixture was stirred under a hydrogen atmosphere at 800 psi (54.4 atm) overnight. The mixture was then filtered through Celite and the filter cake was washed thoroughly with EtOAc. The filtrate was concentrated and the residue purified by flash chromatography to afford 36 mg (5%) of methyl 2{trans-4-(4-(4-amino-5-oxo-7,7-dihydropyrido[4,3-a]pyrimidin-6(5H)-yl)phenyl}cyclohexyl)acetate.

¹H NMR (300 MHz, CDCl₃): δ ppm 1.05-1.22 (m, 2H), 1.40-1.55 (m, 2H), 1.80-1.95 (m, 5H), 2.21-2.26 (m, 2H), 2.40-2.50 (m, 1H), 2.55-2.65 (m, 2H), 3.66-3.73 (m, 5H), 4.64 (s, 2H), 7.11-7.21 (m, 4H).

### Preparation of Examples

### Example 1

### Preparation of {4-[4-(4-amino-2-methoxy-5-oxo-7,8-dihydropyrido[4,3-d]pyrimidin-6(5H)-yl-3-fluorophenyl]cyclohexyl}acetic acid

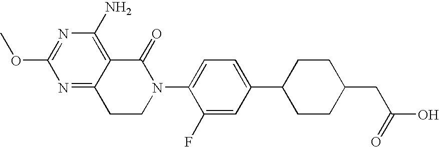

Lithium hydroxide (4.3 mg, 0.18 mmol) was added to a solution of methyl{4-[4-(4-amino-2-methoxy-5-oxo-7,8-dihydropyrido[4,3-d]pyrimidin-6(5H)-yl)-3-fluorophenyl]cyclohexyl}acetate (20 mg, 0.045 mmol) in THF/MeOH/water (2 mL; 3:2:1). The resulting solution was stirred at room temperature for 4 hours. 1M HCl was added to the reaction solution to adjust the pH to about 3.20% isopropanol in DCM was added to the reaction mixture. The organic layer was collected and dried. The solid was purified by chromatography (4 g silica gel column 1-10% MeOH:DCM) to give {4-[4-(4-amino-2-methoxy-5-oxo-7,8-dihydropyrido[4,3-d]pyrimidin-6(5H)-yl)-3-fluorophenyl]cyclohexyl}acetic acid (1-A) as an off white solid (8 mg, 40% yield).

¹H NMR (400 MHz, MeOH-d₄) δ ppm 1.09-1.27 (m, 1H), 1.42-1.56 (m, 1H), 1.62-1.74 (m, 4H), 1.77-1.95 (m, 2H), 2.20 (d, J=7.06 Hz, 1H), 2.40 (d, J=7.48 Hz, 1H), 2.46-2.56 (m, 1H), 2.56-2.67 (m, 1H), 3.00 (t, J=6.85 Hz, 2H), 3.81-3.88 (m, 2H) 3.92 (s, 3H), 6.99-7.13 (m, 2H), 7.20-7.29 (m, 1H). m/z=429.5 (M+1).

### Example 2

### Preparation of {trans-4-[4-(4-amino-2-methoxy-5-oxo-7,8-dihydropyrido[4,3-d]pyrimidin-6(5H)-yl)phenyl]cyclohexyl}acetic acid (2-A)

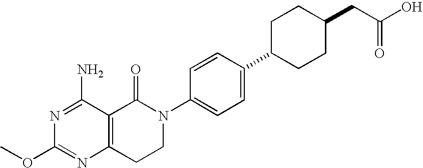

Lithium hydroxide (12.6 mg, 0.3 mmol) was added to a solution of methyl{4-[4-(4-amino-2-methoxy-5-oxo-7,8-dihydropyrido[4,3-a]pyrimidin-6(5H)-yl)phenyl]cyclohexyl}acetate (14 mg, 0.033 mmol) in THF/MeOH/water (2:2:0.3 mL) The resulting solution was stirred at +50° C. for 18 hours. The reaction was concentrated to dryness and 0.5M citric acid (2 mL) and water (4 mL) were added. The suspension was stirred at room temperature for 20 minutes. The solid was filtered off, washed with water and dried on the high vacuum at 45° C. to obtain {trans-4-[4-(4-amino-2-methoxy-5-oxo-7,8-dihydropyrido[4,3-d]pyrimidin-6(5H)-yl)phenyl]cyclohexyl}acetic acid as a white solid (6.8 mg, 50% yield).

¹H NMR (500 MHz, DMSO-d₆) δ ppm 1.08-1.18 (m, 2H), 1.39-1.53 (m, 2H), 1.67-1.86 (m, 6H), 2.14 (d, 2H), 2.94 (t, 2H), 3.81-3.91 (m, 5H), 7.17-7.31 (m, 4H), 7.79 (s, 1H), 8.36 (s, 1H), 12.05 (s, 1H). m/z=411.0 (M+1).

### Preparation of trans-4-[4-(4-amino-2-methoxy-5-oxo-7,8-dihydropyrido[4,3-d]pyrimidin-6(5H)-yl)phenyl]cyclohexyl}acetic acid hydrochloride (2-B)

To a mixture of {trans-4-[4-(4-amino-2-methoxy-5-oxo-7,8-dihydropyrido[4,3-d]pyrimidin-6(5H)-yl)phenyl]cyclohexyl}acetic acid (2A: 60 mg, 0.15 mmol) and MeOH (3 mL) was added 1.0M HCl (0.165 mL, 0.165 mmol) which was allowed to stir at room temperature. The mixture was concentrated at +35° C. and dried under vacuum to give {trans-4-[4-(4-amino-2-methoxy-5-oxo-7,8-dihydropyrido[4,3-d]pyrimidin-6(5H)-yl)phenyl]cyclohexyl}acetic acid hydrochloride (65 mg, 100% yield). m/z=411.0 (M+1)

### Preparation of Potassium {trans-4-[4-(4-amino-2-methoxy-5-oxo-7,8-dihydropyrido[4,3-d]pyrimidin-6(5H)-yl)phenyl]cyclohexyl}acetate salt (2-C)

To a round-bottomed flask was added methyl {trans-4-(4-(4-amino-2-methoxy-5-oxo-7,8-dihydropyrido[4,3-d]pyrimidin-6(5H)-yl)phenyl]cyclohexyl}acetate (2-A: 119.0 g, 280.3 mmol), MeOH (357 mL), THF (714 mL) and 1M potassium hydroxide (874.63 g, 840.9 mmol). The slurry was heated to reflux for 30 minutes. The mixture was filtered through Celite with MeOH washing (100 mL). The combined filtrate layers were concentrated on rotoevaporator to remove all MeOH/THF. The solid product was filtered and washed with water. The cake was dried in a vacuum oven (50° C.) for 18 hours to give potassium {trans-4-[4-(4-amino-2-methoxy-5-oxo-7,8-dihydropyrido[4,3-d]pyrimidin-6(5H)-yl)phenyl]cyclohexyl}acetate salt as white needles (85 g, 67.6% yield).

¹H NMR (400 MHz, DMSO-d₆) δ ppm 0.86-1.04 (m, 2H) 1.28-1.46 (m, 2H) 1.59 (br. s., 1H) 1.64-1.87 (m, 6H) 2.32-2.45 (m, 1H) 2.90 (t, J=6.73 Hz, 2H) 3.77-3.93 (m, 5H) 7.20 (s, 4H) 7.74 (d, J=3.51 Hz, 1H) 8.34 (d, J=3.71 Hz, 1H). m/z=411.0 (M+1)

### Preparation of Sodium {trans-4-[4-(4-amino-2-methoxy-5-oxo-7,8-dihydropyrido[4,3-d]pyrimidin-6(5H)-yl)phenyl]cyclohexyl}acetate salt (2-D)

To a mixture of {trans-4-[4-(4-amino-2-methoxy-5-oxo-7,8-dihydropyrido[4,3-d]pyrimidin-6(5H)-yl)phenyl]cyclohexyl}acetic acid (2-A: 60 mg, 0.15 mmol) and MeOH (3 mL) was added 1.0M NaOH (0.165 mL, 0.165 mmol) which was allowed to stir at room temperature. The mixture was concentrated at +35° C. and dried in vacuo to give Sodium {trans-4-[4-(4-amino-2-methoxy-5-oxo-7,8-dihydropyrido[4,3-d]pyrimidin-6(5H)-yl)phenyl]cyclohexyl}acetic acid salt (65 mg, 100% yield). m/z=411.0 (M+1)

### Example 3

### Preparation of (trans-4-{4-[(7R)-4-amino-2-methoxy-7-methyl-5-oxo-7,8-dihydropyrido[4,3-d]pyrimidin-6(5H)-yl]phenyl}cyclohexyl)acetic acid

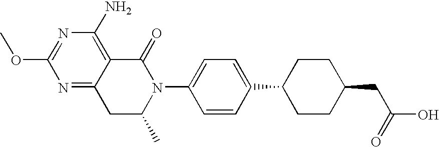

The title compound was prepared according to the procedures outlined in Example 1 using methyl(trans-4-{(4-[(7R)-4-amino-2-methoxy-7-methyl-5-oxo-7,8-dihydropyrido[4,3-d]pyrimidin-6(5H)-yl]phenyl}cyclohexyl)acetate (130 mg, 0.296 mmol) to provide an off-white solid (65 mgs, 52% yield). The resulting racemic mixture was separated on a Chiralcel OJ-H column (70:30 of CO₂:MeOH at 10 g/min) to afford trans-4-{4-[(7R)-4-amino-2-methoxy-7-methyl-5-oxo-7,8-dihydropyrido[4,3-d]pyrimidin-6(5H)-yl]phenyl}cyclohexyl)acetic acid (3 mg, ret time=2.94 minutes).

¹H NMR (400 MHz, MeOH-d₄) δ ppm 1.11-1.25 (m, 5H), 1.47-1.62 (m, 2H), 1.77-1.98 (m, 5H), 2.20 (d, J=6.65 Hz, 2H), 2.53 (t, J=12.25 Hz, 1H), 2.71 (dd, J=16.62, 3.74 Hz, 1H), 3.38 (dd, J=16.82, 6.44 Hz, 1H), 3.93 (s, 3H), 4.07-4.19 (m, 1H), 7.20-7.26 (m, 2H), 7.27-7.34 (m, 2H). m/z=425.5 (M+1).

### Example 4

### Preparation of (trans-4-{4-[(7S)-4-amino-2-methoxy-7-methyl-5-oxo-7,8-dihydropyrido[4,3-d]pyrimidin-6(5H)-yl]phenyl}cyclohexyl)acetic acid

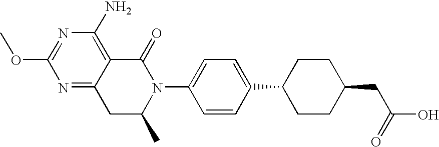

The title compound was prepared according to the procedures outlined in Example 1 using methyl(trans-4-{4-[(7R)-4-amino-2-methoxy-7-methyl-5-oxo-7,8-dihydropyrido[4,3-d]pyrimidin-6(5H)-yl]phenyl}cyclohexyl)acetate (130 mg, 0.296 mmol) to provide the acid (65 mgs, 52% yield). The resulting racemic mixture was separated on a Chiralcel OJ-H column (70:30 of CO₂:MeOH at 10 g/min) to give (trans-4-{4-[(7S)-4-amino-2-methoxy-7-methyl-5-oxo-7,8-dihydropyrido[4,3-d]pyrimidin-6(5H)-yl]phenyl}cyclohexyl)acetic acid (3 mg, ret. time=3.72 minutes).

¹H NMR (400 MHz, MeOH-d₄) δ ppm 1.11-1.25 (m, 5H), 1.47-1.62 (m, 2H), 1.77-1.98 (m, 5H), 2.20 (d, J=6.65 Hz, 2H), 2.53 (t, J=12.25 Hz, 1H), 2.71 (dd, J=16.62, 3.74 Hz, 1H), 3.38 (dd, J=16.82, 6.44 Hz, 1H), 3.93 (s, 3H), 4.07-4.19 (m, 1H), 7.20-7.26 (m, 2H), 7.27-7.34 (m, 2H). m/z=425.5 (M+1).

### Example 5

### Preparation of {trans-4-[4-(4-amino-2-methoxy-5-oxo-7,8-dihydropyrido[4,3-d]pyrimidin-6(5H)-yl)-2-methylphenyl]cyclohexyl}acetic acid

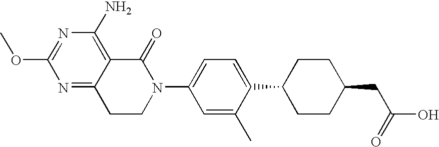

The title compound was prepared according to the procedures outlined in Example 2 using methyl{trans-4-[4-(4-amino-2-methoxy-5-oxo-7,8-dihydropyrido[4,3-d]pyrimidin-6(5H)-yl)-2-methylphenyl]cyclohexyl}acetate (62 mg, 0.14 mmol) to provide {trans-4-[4-(4-amino-2-methoxy-5-oxo-7,8-dihydropyrido[4,3-d]pyrimidin-6(5H)-yl)-2-methylphenyl]cyclohexyl}acetic acid (33 mg, 55%.yield).

¹H NMR (500 MHz, DMSO-d₆) δ ppm 0.85-1.30 (m, 2H), 1.28-1.56 (m, 2H), 1.60-1.94 (m, 5H), 2.12 (d, J=6.65 Hz, 2H), 2.25 (s, 3H), 2.54-2.68 (m, 1H), 2.89 (t, J=6.65 Hz, 2H), 3.61-3.96 (m, 5H), 7.07 (s, 2H). m/z=425.5 (M+1).

### Example 6

### Preparation of {trans-4-[4-(4-amino-2-methoxy-5-oxo-7,8-dihydropyrido[4,3-d]pyrimidin-6(5H)-yl)-2-chlorophenyl]cyclohexyl}acetic acid

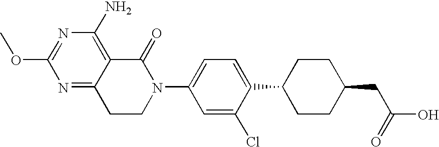

The title compound was prepared according to the procedures outlined in Example 2 using methyl{trans-4-[4-(4-amino-2-methoxy-5-oxo-7,8-dihydropyrido[4,3-d]pyrimidin-6(5H)-yl)-2-chlorophenyl]cyclohexyl}acetate (50.5 mg, 0.11) to provide trans-4-[4-(4-amino-2-methoxy-5-oxo-7,8-dihydropyrido[4,3-d]pyrimidin-6(5H)-yl)-2-chlorophenyl]cyclohexyl}acetic acid (8 mg, 20% yield).

¹H NMR (500 MHz, DMSO-d₆) δ ppm 1.01-1.19 (m, 2H), 1.38-1.54 (m, 2H), 1.64-1.89 (m, 5H) 2.13 (d, J=6.65 Hz, 2H), 2.85 (t, J=8.72 Hz, 1H), 2.91 (t, J=6.85 Hz, 2H), 3.82 (s, 3H), 3.86 (t, J=6.65 Hz, 2H), 7.26 (d, J=8.31 Hz, 1H), 7.34-7.45 (m, 2H). m/z=445.5 (M+1).

### Example 7

### Preparation of {trans-4-[4-(4-amino-2-methoxy-7-methyl-5-oxo-7,8-dihydropyrido[4,3-d]pyrimidin-6(5H)-yl)-2-fluorophenyl]cyclohexyl}acetic acid

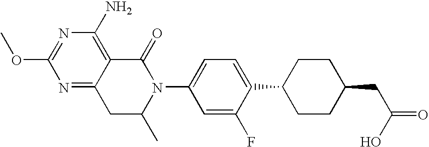

The title compound was prepared according to the procedures outlined in Example 1 using methyl{trans-4-[4-(4-amino-2-methoxy-7-methyl-5-oxo-7,8-dihydropyrido[4,3-d]pyrimidin-6(5H)-yl)-2-fluorophenyl]cyclohexyl}acetate (80 mg, 0.18 mmol) to provide {trans-4-[4-(4-amino-2-methoxy-7-methyl-5-oxo-7,8-dihydropyrido[4,3-d]pyrimidin-6(5H)-yl)-2-fluorophenyl]cyclohexyl}acetic acid (25 mg, 32% yield).

¹H NMR (500 MHz, MeOH-d₄) δ ppm 1.11-1.28 (m, 5H), 1.47-1.63 (m, 2H), 1.76-1.96 (m, 5H), 2.20 (d, J=7.06 Hz, 2H), 2.71 (dd, J=17.03, 3.74 Hz, 1H), 2.76-2.89 (m, 1H), 3.31-3.38 (m, 1H), 3.92 (s, 3H), 4.06-4.18 (m, 1H), 7.00 (dd, J=17.65, 8.52 Hz, 2H), 7.29 (t, J=8.31 Hz, 1H). m/z=443.5 (M+1).

### Example 8

### Preparation of {trans-4-[4-(4-amino-2-methoxy-5-oxo-7,8-dihydropyrido[4,3-d]pyrimidin-6(5H)-yl)-2-fluorophenyl]cyclohexyl}acetic acid

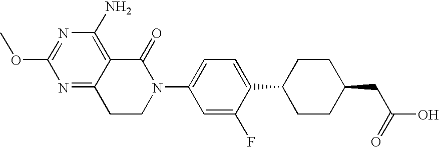

The title compound was prepared according to procedures outlined in Example 1 from methyl{trans-4-[4-(4-amino-2-methoxy-5-oxo-7,8-dihydropyrido[4,3-d]pyrimidin-6(5H)-yl)-2-fluorophenyl]cyclohexyl}acetate (150 mg, 0.339 mmol) to provide {trans-4-[4-(4-amino-2-methoxy-5-oxo-7,8-dihydropyrido[4,3-d]pyrimidin-6(5H)-yl)-2-fluorophenyl]cyclohexyl}acetic acid (70 mg, 36% yield).

¹H NMR (400 MHz, DMSO-d₆) δ ppm 1.01-1.18 (m, 2H), 1.49 (q, J=12.19 Hz, 2H), 1.58-1.87 (m, 5H), 2.12 (d, J=7.06 Hz, 2H), 2.65-2.80 (m, 1H), 2.91 (t, J=6.85 Hz, 2H), 3.73-3.91 (m, 5H), 7.06-7.21 (m, 2H), 7.32 (t, J=8.52 Hz, 1H), 7.82 (d, J=4.15 Hz, 1H), 8.30 (d, J=4.15 Hz, 1H), 12.03 (br. s., 1H). m/z=395.4 (M+1).

### Example 9

### Preparation of {trans-4-[4-(4-amino-2-isopropoxy-5-oxo-7,8-dihydropyrido[4,3-d]pyrimidin-6(5H)-yl)phenyl]cyclohexyl}acetic acid

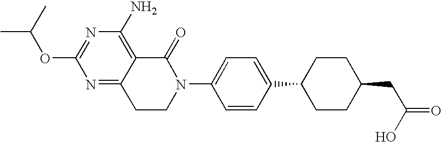

The title compound was prepared according to the procedures outlined in Example 2 from methyl{trans-4-[4-(4-amino-2-isopropoxy-5-oxo-7,8-dihydropyrido[4,3-d]pyrimidin-6(5H)-yl)phenyl]cyclohexyl}acetate (10.4 mg, 0.023 mmol) to provide {trans-4-[4-(4-amino-2-isopropoxy-5-oxo-7,8-dihydropyrido[4,3-d]pyrimidin-6(5H)-yl)phenyl]cyclohexyl}acetic acid (8.5 mg, 84% yield).

¹H NMR (500 MHz, DMSO-d₆) δ ppm 1.03-1.19 (m, 2H), 1.28 (d, 6H), 1.39-1.54 (m, 2H), 1.66-1.87 (m, 6H), 2.14 (d, 2H), 2.90 (t, 2H), 3.85 (t, 2H), 5.13-5.24 (m, 1H), 7.17-7.29 (m, 4H), 7.69 (d, 1H), 8.33 (d, 1H), 12.05 (s, 1H). m/z=439.0.

### Example 10

### Preparation of (trans-4-{4-[4-amino-2-(2-methoxyethoxy)-5-oxo-7,8-dihyropyrido[4,3-d]pyrimidin-6(5H)-yl]phenyl}cyclohexyl)acetic acid

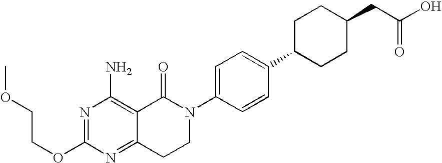

The title compound was prepared according to the procedures outlined in Example 2 from methyl(trans-4-{4-[4-amino-2-(2-methoxyethoxy)-5-oxo-7,8-dihydropyrido[4,3-d]pyrimidin-6(5H)-yl]phenyl}cyclohexyl)acetate (14.1 mg, 0.03 mmol) to provide (trans-4-{4-[4-amino-2-(2-methoxyethoxy)-5-oxo-7,8-dihydropyrido[4,3-d]pyrimidin-6(5H)-yl]phenyl}cyclohexyl)acetic acid (5 mg, 40%).

m/z=455.0 (M+1)

### Example 11

### Preparation of {trans-4-[4-(4-amino-2-ethoxy-5-oxo-7,8-dihydropyrido[4,3-d]pyrimidin-6(5H)-yl)phenyl]cyclohexyl}acetic acid

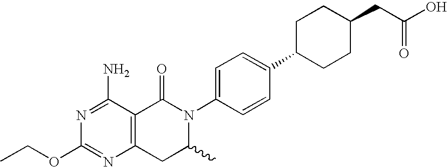

The title compound was prepared according to the procedures outlined in Example 2 from methyl{trans-4-[4-(4-amino-2-ethoxy-5-oxo-7,8-dihydropyrido[4,3-d]pyrimidin-6(5H)-yl)phenyl]cyclohexyl}acetate (11 mg, 0.025 mmol) to provide {trans-4-[4-(4-amino-2-ethoxy-5-oxo-7,8-dihydropyrido[4,3-d]pyrimidin-6(5H)-yl)phenyl]cyclohexyl}acetic acid (8 mg, 80% yield).

¹H NMR (500 MHz, DMSO-d₆) δ ppm 1.07-1.19 (m, 2H), 1.28 (t, 3H), 1.39-1.54 (m, 2H), 1.68-1.85 (m, 6H), 2.14 (d, 2H), 2.91 (t, 2H), 3.84 (t, 2H), 4.29 (q, 2H), 7.21-7.29 (m, 4H), 7.73 (d, 1H), 8.35 (d, 1H), 12.05 (s, 1H). m/z=425.0 (M+1).

### Example 12

### Preparation of {trans-4-[4-(4-amino-2-methoxy-7-methyl-5-oxo-7,8-dihydropyrido[4,3-d]pyrimidin-6(5H)-yl)phenyl]cyclohexyl}acetic acid

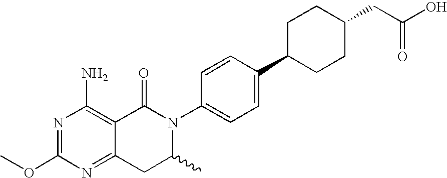

The title compound was prepared according to the procedures outlined in Example 1 using methyl (trans-4-[4-(4-amino-2-methoxy-7-methyl-5-oxo-7,8-dihydropyrido[4,3-d]pyrimidin-6(5H)-yl)phenyl]cyclohexyl)acetate (130 mg, 0.30 mmol) to provide 2-(trans-4-[4-(4-amino-2-methoxy-7-methyl-5-oxo-7,8-dihydropyrido[4,3-d]pyrimidin-6(5H)-yl)phenyl]cyclohexyl)acetic acid (65 mg, 52% yield).

¹H NMR (400 MHz, METHANOL-d4) d ppm 1.08-1.28 (m, 5H) 1.40-1.59 (m, 2H) 1.73-1.97 (m, 5H) 2.20 (d, J=6.65 Hz, 2H) 2.49 (t, J=12.05 Hz, 1H) 2.70 (dd, J=16.62, 3.74 Hz, 1H) 3.30-3.39 (m, 1H) 3.92 (s, 3H) 4.02-4.15 (m, 1H) 7.13-7.21 (m, 2H) 7.22-7.32 (m, 2H). m/z=425.5 (M+1).

### Example 13

### Preparation of 2-(4-(4-(4-amino-2-methyl-5-oxo-7,8-dihydropyrido[4,3-d]pyrimidin-6(5H)-yl)phenyl)cyclohexyl)acetic acid

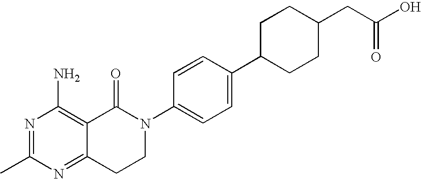

The title compound was prepared according to the procedures outlined in Example 1 using methyl-2-(4-(4-(4-amino-2-methyl-5-oxo-7,8-dihydropyrido[4,3-d]pyrimidin-6(5H)-yl)phenyl)cyclohexyl)acetate (20 mg, 0.049 mmol) to provide 2-(4-(4-(4-amino-2-methyl-5-oxo-7,8-dihydropyrido[4,3-d]pyrimidin-6(5H)-yl)phenyl)cyclohexyl)acetic acid (19 mg, 0.049 mmol).

¹H NMR (400 MHz, METHANOL-d₄) δ ppm 1.08-1.32 (m, 1H) 1.44-1.59 (m, 1H) 1.61-1.77 (m, 4H) 1.77-1.96 (m, 3H) 2.20 (d, J=6.64 Hz, 2H) 2.43 (s, 3H) 2.45-2.67 (m, 2H) 3.04 (t, J=7.42 Hz, 2H) 3.87-4.00 (m, 2H) 7.15-7.40 (m, 4H). m/z=395.4 (M+1).

### Example 14

### Preparation of {trans-4-[4-(4-amino-2-methyl-5-oxo-7,8-dihydropyrido[4,3-d]pyrimidin-6(5H)-yl)phenyl]cyclohexyl}acetic acid

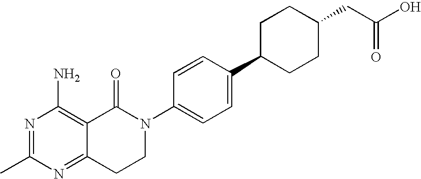

The title compound was prepared according to the procedures outlined in Example 1 using methyl{trans-4-[4-(4-amino-2-methyl-5-oxo-7,8-dihydropyrido[4,3-d]pyrimidin-6(5H)-yl)phenyl]cyclohexyl}acetate (60 mg, 0.15 mmol) to provide methyl{trans-4-[4-(4-amino-2-methyl-5-oxo-7,8-dihydropyrido[4,3-d]pyrimidin-6(5H)-yl)phenyl]cyclohexyl}acetic acid (52 mg, 90% yield).

¹H NMR (500 MHz, DMSO-d₆) 8 ppm 1.08-1.28 (m, 2H), 1.44-1.64 (m, 2H), 1.75-1.97 (m, 5H), 2.21 (d, J=7.06 Hz, 2H), 2.42-2.58 (m, 4H), 3.18 (t, J=6.85 Hz, 2H), 4.01 (t, J=6.85 Hz, 2H), 7.28 (q, J=8.59 Hz, 4H). m/z=395.1 (M+1).

### Example 15

### Preparation of {cis-4-[4-(4-amino-2-methyl-5-oxo-7,8-dihydropyrido[4,3-d]pyrimidin-6(5H)-yl)phenyl]cyclohexyl}acetic acid

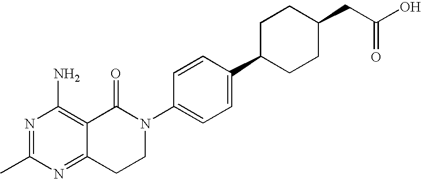

The title compound was prepared according to the procedures outlined in Example 1 using methyl{cis-4-[4-(4-amino-2-methyl-5-oxo-7,8-dihydropyrido[4,3-d]pyrimidin-6(5H)-yl)phenyl]cyclohexyl}acetate (50 mg, 0.12 mmol) to provide {cis-4-[4-(4-amino-2-methyl-5-oxo-7,8-dihydropyrido[4,3-d]pyrimidin-6(5H)-yl)phenyl]cyclohexyl}acetic acid (40 mg, 83% yield).

¹H NMR (400 MHz, MeOH-d₄) δ ppm 1.61-1.78 (m, 8H), 2.20-2.30 (m, 1H), 2.39-2.47 (m, 5H), 2.54-2.66 (m, 1H), 3.06 (t, J=6.85 Hz, 2H), 3.95 (t, J=6.85 Hz, 2H), 7.28 (dd, 4H). m/z=395.1 (M+1).

### Example 16

### Preparation of {trans-4-[4-(4-amino-2,7-dimethyl-5-oxo-7,8-dihydropyrido[4,3-d]pyrimidin-6(5H)-yl)phenyl]cyclohexyl}acetic acid

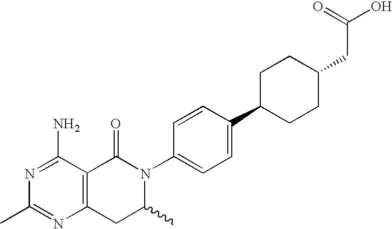

To a solution of methyl{trans-4-[4-(4-amino-2,7-dimethyl-5-oxo-7,8-dihydropyrido[4,3-d]pyrimidin-6(5H)-yl)phenyl]cyclohexyl}acetate (25 mg, 0.059 mmol) in MeOH (1.5 mL). To the solution was added a 2M solution of NaOH (0.75 mL). The reaction was stirred for 2 days. The solvent was removed under reduced pressure and acidified with 1M citric acid. The solid was filtered and dried for 4 hours in vacuo at 50° C. The white solid was purified using reverse phase preparatory HPLC to give {trans-4-[4-(4-amino-2,7-dimethyl-5-oxo-7,8-dihydropyrido[4,3-d]pyrimidin-6(5H)-yl)phenyl]cyclohexyl}acetic acid (4 mg, 20% yield).

¹H NMR (400 MHz, Chloroform-d) δ ppm 1.13-1.24 (m, 2H), 1.26 (d, J=6.64 Hz, 3H), 1.45-1.60 (m, 2H), 1.83-2.02 (m, 5H), 2.30 (d, J=6.83 Hz, 2H), 2.52 (br. S., 4H), 2.84 (dd, J=16.69, 3.61 Hz, 1H), 3.40 (dd, J=16.79, 6.25 Hz, 1H), 4.08-4.18 (m, 1H), 6.33 (br. S., 1H), 7.23-7.31 (m, 2H), 8.62 (br. s. 1H).

### Example 17

### Preparation of {trans-4-[4-(4-amino-2-ethyl-5-oxo-7,8-dihydropyrido[4,3-d]pyrimidin-6(5H)-yl)phenyl]cyclohexyl}acetic acid

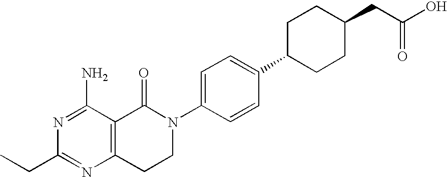

The title compound was prepared according to the procedures outlined in Example 1 using methyl {trans-4-[4-(4-amino-2-ethyl-5-oxo-7,8-dihydropyrido[4,3-α]pyrimidin-6(5H)-yl)phenyl]cyclohexyl}acetate: (26 mg, 0.062 mmol) to provide {trans-4-[4-(4-amino-2-ethyl-5-oxo-7,8-dihydropyrido[4,3-d]pyrimidin-6(5H)-yl)phenyl]cyclohexyl}acetic acid: (15 mg, 57% yield).

¹H NMR (500 MHz, DMSO-d6) d ppm 1.08-1.14 (m, 2H) 1.22 (t, 3H) 1.42-1.51 (m, 2H) 1.69-1.85 (m, 5H) 2.13 (d, 2H) 2.43-2.48 (m, 1H) 2.62 (q, 2H) 2.97 (t, 2H) 3.88 (t, 2H) 7.21-7.32 (m, 4H) 7.73 (s, 1H) 8.25 (s, 1H) 12.06 (s, 1H)

### Example 18

### Preparation of {trans-4-[4-(4-amino-5-oxo-7,8-dihydropyrido[4,3-d]pyrimidin-6(5H)-yl)phenyl]cyclohexyl}acetic acid

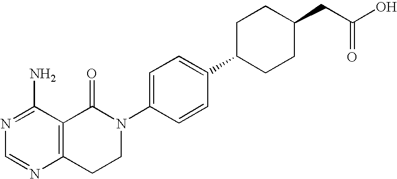

A solution of lithium hydroxide (2.0M in water, 3.36 ml) was added to a solution of methyl 2-(trans-4-(4-(4-amino-5-oxo-7,7-dihydropyrido[4,3-α]pyrimidin-6(5H)-yl)phenyl)cyclohexyl)acetate (265 mg, 0.672 mol) in dioxane (25 mL) and the mixture was stirred at 45 C for 4 hours. After cooling to ambient temperature the volume of the reaction mixture was reduced to 5 mL in vacuo and water (5 mL) was added. The solution was acidified to approximately pH 2-3 with 10% aqueous citric acid and the resulting precipitate collected by filtration. The collected solid was washed with water, methanol and finally dichloromethane to give the title compound as a white solid (251 mg, 98% yield).

¹H NMR 1.00-1.15 (m, 2H), 1.38-1.50 (m, 2H), 1.62-1.83 (m, 5H), 2.08-2.13 (m, 2H), 2.40-2.50 (m 1H), 2.97 (t, 2H), 3.87 (t, 2H), 7.18-7.28 (m, 4H), 7.56 (bs, NH), 8.24 (bs, NH), 8.34 (s, 1H).

### Pharmacological Testing

The practice of the invention for the treatment of diseases modulated by the inhibition of DGAT-1 can be evidenced by activity in at least one of the protocols described hereinbelow.

### In Vitro Assay for Inhibition of DGAT-1 Activity

Human full-length diacylglycerol:acylCoA acyltransferase 1 (DGAT-1) was expressed in Sf9 insect cells which are then lysed and a crude membrane fraction (105,000×g pellet) was prepared. The DGAT-1 gene is a human DGAT-1 gene described in J Biol Chem 273:26765 (1998) and U.S. Pat. No. 6,100,077.

In vitro inhibition of DGAT-1 was measured using a modification, further described below, of the assay methodology described in U.S. Pat. No. 6,994,956 B2. The cells were cultured as follows. Sf9 cells (20 L) were infected with 4 mL of DGAT-1 Baculovirus Infected Insect Cells (BIIC) for 72 hours in a Wave Bioreactor System 20/50P (Wave Biotec/GE Healthcare).

Crude DGAT-1 microsomes were prepared as follows. Cell pellets were washed once with ice-cold Dulbecco's phosphate-buffered saline. Cells were collected in tabletop centrifuge (Beckman GS-6KR), 15 minutes, 2000×g, 4° C. Twenty (20) mL of ice-cold Microsome Buffer (MB) was added per 5 g of cell pellet. The suspension was passed through a microfluidizer 3 times (18K psi). The lysate was transferred to centrifuge tubes and centrifuged for 20 minutes at 5000×g (Beckman-Coulter, Inc. Allegra® 64R High-Speed Refrigerated Benchtop Centrifuge, F0650 rotor) at 4° C. The supernatant was transferred to ultracentrifuge tubes and centrifuged at 125,000×g for 1 hour in a Beckman Ti-45 rotor, 4° C. The supernatant fluid was discarded. The pellet was resuspended in 70 mL of MB by sonication. The microsome concentration was determined using Bio-Rad Protein DC Protein Assay. The samples were portioned, flash frozen and stored at −80° C.

The Microsome Buffer, used for microsome preparation, was prepared by conventional means and contained 125 mM sucrose, 3 mM imidazole, 0.2 μg/mL aprotinin, 0.2 μg/mL leupeptin and 5 mM dithiothreitol (Cleland's reagent) at pH-7.4 DGAT-1 activity was measured in 384-well format in a total assay volume of 20 μl that contained, Hepes buffer (50 mM, pH 7.5), MgCl₂ (10 mM), bovine serum albumin (0.6 mg/ml), [¹⁴C]decanoylCoA (25 μM, 58 Ci/mol) and microsomes (5.6 μg/ml) into which 1,2 dioleoyl-sn-glycerol (75 μM) in acetone has already been incorporated. Inhibitors in DMSO were pre-incubated with membranes before initiating the DGAT-1 reaction by the addition of decanoylCoA. Two control DGAT-1 reactions were also incubated in parallel: 1) DMSO without inhibitor to measure zero percent effect of inhibition and 2) and a maximally inhibited DGAT-1 reaction (“blank”) incubated with 1 μM {trans-4-[4-(4-amino-2,7,7-trimethyl-7H-pyrimido[4,5-b][1,4]oxazin-6-yl)phenyl]cyclohexyl}acetic acid (WO2004/047755), which was the 100 percent effect sample. The concentration of DMSO in the reaction mix was 2.5%. The inhibitors were present at a range of eight concentrations to generate an apparent IC₅₀ for each compound. The eight inhibitor concentration employed ranged from 3 μM to 1 nM (from high to low concentration). Specifically, the eight concentrations used were 3 μM, 1 μM, 300 nM, 100 nM, 30 nM, 10 nM, 3 nM and 1 nM.

The reactions were allowed to proceed for 1.5 h at room temperature and then terminated by the addition of 20 μl of EDTA (40 mM). Reaction mixture is then mixed by trituration with 30 μl of Microscint™-E (Perkin Elmer). Plates contents were allowed to partition for 15 to 30 min before ¹⁴C was measured in a scintillation spectrometer (Wallac Microbeta Trilux 1450-030, 12 detector in the top-count DPM mode). Percent inhibition of test compounds was computed as 100-((DPM DMSO uninhibited-DPM test compound)/(DPM DMSO uninhibited)).

Four separate assays were performed using 4 different methods of analysis. The method of analysis of Assay 1 was the same as Assay 4 (described above) except microsomes were utilized at 25 μg/mL instead of 5 μg/mL. The method of analysis of Assay 2 was the same as Assay 4 (described above) except eleven (11) concentrations of inhibitor were employed instead of eight (8). The method of analysis of Assay 3 was the same as Assay 2 except the compounds were serially diluted in a different laboratory.

Exemplary compounds of the invention, described in Examples above were tested for in vitro DGAT-1 inhibition, and were found to exhibit DGAT-1 inhibition with IC₅₀ values set forth in Table 1. Where this DGAT-1 inhibition assay was performed on a compound more than once, an average is provided for that compound.

TABLE 1

| Example | | Assay | Assay | Assay | Assay |
|---|---|---|---|---|---|
| No. | Compound Name | 1 | 2 | 3 | 4 |
| 1 | {4-[4-(4-amino-2-methoxy-5-oxo-7,8- | | 0.0016 | | |
| | dihydropyrido[4,3-d]pyrimidin-6(5H)- | | n = 4 | | |
| | yl)-3-fluorophenyl]cyclohexyl}acetic | | | | |
| | acid | | | | |
| 2-A | {trans-4-[4-(4-amino-2-methoxy-5- | | 0.00156 | 0.00917 | 0.00359 |
| | oxo-7,8-dihydropyrido[4,3-d]pyrimidin- | | n = 4 | n = 2 | n = 2 |
| | 6(5H)-yl)phenyl]cyclohexyl}acetic acid | | | | |
| 2-B | {trans-4-[4-(4-amino-2-methoxy-5- | | 0.0239 | 0.0142 | |
| | oxo-7,8-dihydropyrido[4,3-d]pyrimidin- | | | | |
| | 6(5H)-yl)phenyl]cyclohexyl}acetic acid | | | | |
| | hydrochloride | | | | |
| 2-C | Potassium {trans-4-[4-(4-amino-2- | | 0.00663 | 0.0387 | |
| | methoxy-5-oxo-7,8-dihydropyrido[4,3- | | n = 2 | n = 2 | |
| | d]pyrimidin-6(5H)- | | | | |
| | yl)phenyl]cyclohexyl}acetate salt | | | | |
| 2-D | Sodium {trans-4-[4-(4-amino-2- | | 0.00429 | | |
| | methoxy-5-oxo-7,8-dihydropyrido[4,3- | | n = 3 | | |
| | d]pyrimidin-6(5H)-yl)phenyl]- | | | | |
| | cyclohexyl}acetate salt | | | | |
| 3 | (trans-4-{4-[(7R)-4-amino-2-methoxy- | | 0.0101 | | |
| | 7-methyl-5-oxo-7,8-dihydropyrido[4,3- | | n = 3 | | |
| | d]pyrimidin-6(5H)- | | | | |
| | yl]phenyl}cyclohexyl)acetic acid | | | | |
| 4 | (trans-4-{4-[(7S)-4-amino-2-methoxy- | | 0.0837 | | |
| | 7-methyl-5-oxo-7,8-dihydropyrido[4,3- | | n = 3 | | |
| | d]pyrimidin-6(5H)- | | | | |
| | yl]phenyl}cyclohexyl)acetic acid | | | | |
| 5 | {trans-4-[4-(4-amino-2-methoxy-5- | | 0.00303 | | |
| | oxo-7,8-dihydropyrido[4,3-d]pyrimidin- | | n = 3 | | |
| | 6(5H)-yl)-2- | | | | |
| | methylphenyl]cyclohexyl}acetic acid | | | | |
| 6 | {trans-4-[4-(4-amino-2-methoxy-5- | | 0.00426 | | |
| | oxo-7,8-dihydropyrido[4,3-d]pyrimidin- | | n = 4 | | |
| | 6(5H)-yl)-2- | | | | |
| | chlorophenyl]cyclohexyl}acetic acid | | | | |
| 7 | {trans-4-[4-(4-amino-2-methoxy-7- | | 0.0165 | 0.0383 | |
| | methyl-5-oxo-7,8-dihydropyrido[4,3- | | | n = 2 | |
| | d]pyrimidin-6(5H)-yl)-2- | | | | |
| | fluorophenyl]cyclohexyl}acetic acid | | | | |
| 8 | {trans-4-[4-(4-amino-2-methoxy-5- | | 0.00379 | 0.0144 | |
| | oxo-7,8-dihydropyrido[4,3-d]pyrimidin- | | | n = 4 | |
| | 6(5H)-yl)-2- | | | | |
| | fluorophenyl]cyclohexyl}acetic acid | | | | |
| 9 | {trans-4-[4-(4-amino-2-isopropoxy-5- | | | 0.183 | 0.142 |
| | oxo-7,8-dihydropyrido[4,3-d]pyrimidin- | | | n = 2 | |
| | 6(5H)-yl)phenyl]cyclohexyl}acetic acid | | | | |
| 10 | (trans-4-{4-[4-amino-2-(2- | | | 0.434 | |
| | methoxyethoxy)-5-oxo-7,8- | | | | |
| | dihydropyrido[4,3-d]pyrimidin-6(5H)- | | | | |
| | yl]phenyl}cyclohexyl)acetic acid | | | | |
| 11 | {trans-4-[4-(4-amino-2-ethoxy-5-oxo- | | | 0.0307 | 0.0207 |
| | 7,8-dihydropyrido[4,3-d]pyrimidin- | | | n = 2 | |
| | 6(5H)-yl)phenyl]cyclohexyl}acetic acid | | | | |
| 12 | {trans-4-[4-(4-amino-2-methoxy-7- | | 0.00687 | 0.0719 | |
| | methyl-5-oxo-7,8-dihydropyrido[4,3- | | | n = 4 | |
| | d]pyrimidin-6(5H)- | | | | |
| | yl)phenyl]cyclohexyl}acetic acid | | | | |
| 13 | {4-[4-(4-amino-2-methyl-5-oxo-7,8- | 0.231 | | | |
| | dihydropyrido[4,3-d]pyrimidin-6(5H)- | n = 3 | | | |
| | yl)phenyl]cyclohexyl}acetic acid | | | | |
| 14 | {trans-4-[4-(4-amino-2-methyl-5-oxo- | 0.618 | | | |
| | 7,8-dihydropyrido[4,3-d]pyrimidin- | n = 2 | | | |
| | 6(5H)-yl)phenyl]cyclohexyl}acetic acid | | | | |
| 15 | {cis-4-[4-(4-amino-2-methyl-5-oxo-7,8- | 0.717 | | | |
| | dihydropyrido[4,3-d]pyrimidin-6(5H)- | n = 2 | | | |
| | yl)phenyl]cyclohexyl}acetic acid | | | | |
| 16 | {trans-4-[4-(4-amino-2,7-dimethyl-5- | | | 0.412 | 0.252 |
| | oxo-7,8-dihydropyrido[4,3-d]pyrimidin- | | | n = 2 | n = 4 |
| | 6(5H)-yl)phenyl]cyclohexyl}acetic acid | | | | |
| 17 | {trans-4-[4-(4-amino-2-ethyl-5-oxo- | | | 0.598 | |
| | 7,8-dihydropyrido[4,3-d]pyrimidin- | | | | |
| | 6(5H)-yl)phenyl]cyclohexyl}acetic acid | | | | |
| 18 | {trans-4-[4-(4-amino-5-oxo-7,8- | | 0.0140 | | |
| | dihydropyrido[4,3-d]pyrimidin-6(5H)- | | n = 2 | | |
| | yl)phenyl]cyclohexyl}acetic acid | | | | |

### In Vivo Assay for Glucose Lowering

Oral glucose tolerance tests (“OGTT”) have been in use in humans since, at least, the 1930s, Pincus et al., Am J Med Sci 188, 782 (1934), and are routinely used in the diagnosis of human diabetes, though not to evaluate the efficacy of therapeutic agents in patients.

KK mice have been used to evaluate glitazones (Fujita, et al., Diabetes. 32, 804-810 (1983); Fujiwara, et al, Diabetes. 37, 1549-48 (1988); Izumi et al. Biopharm Durq Dispos. 18, 247-257 (1997), metformin (Reddi, et al., Diabet Metabl. 19, 44-51 (1993), glucosidase inhibitors (Hamada, et al., Jap Pharmacol Ther, 17, 17-28 (1988); Matsuo, et al., Am J Clin Nutr. 55, 314S-317S (1992)), and the extra-pancreatic effects of sulfonylureas (Kameda, et al., Arzenim Forsch./Drug Res. 32, 39044 (1982); and Muller, et al., Horm Metabl Res, 28, 469-487 (1990)).

KK mice are derived from an inbred line first established by Kondo et al. (Kondo, et al., Bull Exp Anim. 6, 107-112 (1957)). The mice spontaneously develop a hereditary form of polygenic diabetes that progresses to cause renal, retinal and neurological complications analogous to those seen in human diabetic subjects, but they do not require insulin or other medication for survival. Another aspect of the invention is directed to the use of KK mice to evaluate the effects of insulin secretagogue agents in the context of an oral glucose tolerance test.

### In Vivo Assay for Food Intake

The following screen may be used to evaluate the efficacy of test compounds for inhibiting food intake in Sprague-Dawley rats after an overnight fast.

Male Sprague-Dawley rats are individually housed and fed powdered chow. They are maintained on a 12 hour light/dark cycle and received food and water ad libitum. The animals are acclimated to the vivarium for a period of one week before testing is conducted. Testing is completed during the light portion of the cycle.

To conduct the food intake efficacy screen, rats are transferred to individual test cages without food the afternoon prior to testing, and the rats are fasted overnight. After the overnight fast, rats are dosed the following morning with vehicle or test compounds. A known antagonist is dosed (3 mg/kg) as a positive control, and a control group receives vehicle alone (no compound). The test compounds are dosed at ranges between 0.1 and 100 mg/kg depending upon the compound. The standard vehicle is 0.5% (w/v) methylcellulose in water and the standard route of administration is oral. However, different vehicles and routes of administration may be used to accommodate various compounds when required. Food is provided to the rats 30 minutes after dosing and an Oxymax automated food intake system (Columbus Instruments, Columbus, Ohio) is started. Individual rat food intake is recorded continuously at 10-minute intervals for a period of two hours. When required, food intake is recorded manually using an electronic scale; food is weighed every 30 minutes after food is provided up to four hours after food is provided. Compound efficacy is determined by comparing the food intake pattern of compound-treated rats to vehicle and the standard positive control.

Throughout this application, various publications are referenced. The disclosures of these publications in their entireties are hereby incorporated by reference into this application for all purposes.

It will be apparent to those skilled in the art that various modifications and variations can be made in the invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims and the application as a whole.

What is claimed is:

1. A compound having Formula Ia 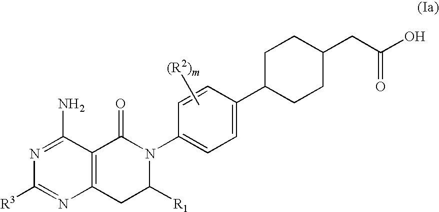
wherein
R¹ is hydrogen (C₁-C₂)alkoxy, halo-substituted (C₁-C₂)alkyl, halo-substituted (C₁-C₂)alkoxy)or (C₁-C₂)alkyl;
each R² is independently halogen, OH, (C₁-C₄)alkyl, cyano, (C₃-C₆)cycloalkyl or (C₁-C₄)alkoxy;
R³ is hydrogen, (C₁-C₂)alkyl, (C₁-C₂)alkoxy, or —O(C₁-C₂)alkyl (C₁-C₂)alkoxy; and
m is 0, 1, 2, or 3;
or a pharmaceutically acceptable salt thereof.

2. A compound according to claim 1 having Formula Ib 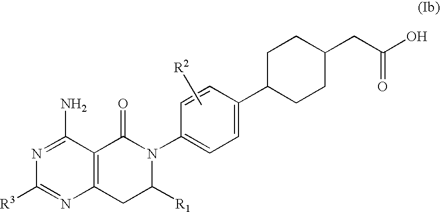
wherein
R¹ is hydrogen or (C₁-C₂)alkyl;
R² is hydrogen, halogen, OH, (C₁-C₄)alkyl, or (C₁-C₄)alkoxy;
R³ is hydrogen or (C₁-C₂)alkoxy;
or a pharmaceutically acceptable salt thereof.

3. A compound according to claim 1 having Formula (II) 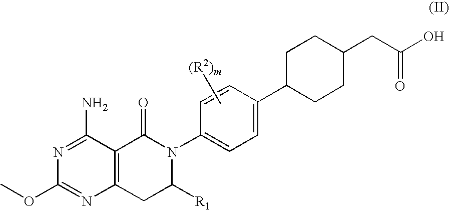
wherein
R¹ is hydrogen;
R² is hydrogen, halogen, OH, (C₁-C₄)alkyl, or (C₁-C₄)alkoxy; and
m is 0, 1, 2, or 3;
or a pharmaceutically acceptable salt thereof.

4. A compound according to claim 1 having Formula (III) 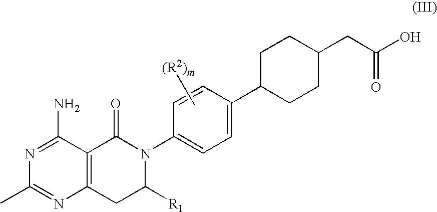
wherein
R¹ is hydrogen;
R² is hydrogen, halogen, OH, (C₁-C₄)alkyl, or (C₁-C₄)alkoxy; and
m is 0, 1, 2, or 3;
or a pharmaceutically acceptable salt thereof.

5. A compound according to claim 1 having Formula (IV) 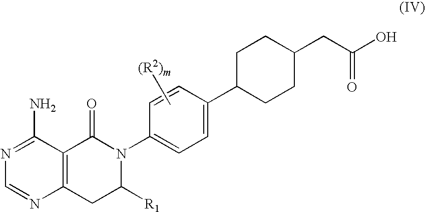
wherein
R¹ is hydrogen,
R² is hydrogen, halogen, (C₁-C₂)alkyl, or (C₁-C₂)alkoxy; and
m is 0, 1, 2, or 3;
or a pharmaceutically acceptable salt thereof.

6. A compound of any of the preceding claims wherein R¹ is hydrogen; R³ is selected from hydrogen, methyl, and methoxy; and m is 1.

7. A compound selected from the group consisting of
{4-[4-(4-amino-2-methoxy-5-oxo-7,8-dihydropyrido[4,3-d]pyrimidin-6(5H)-yl)-3-fluorophenyl]cyclohexyl}acetic acid;
{trans-4-[4-(4-amino-2-methoxy-5-oxo-7,8-dihydropyrido[4,3-d]pyrimidin-6(5H)-yl)phenyl]cyclohexyl}acetic acid;
{trans-4-[4-(4-amino-2-methoxy-5-oxo-7,8-dihydropyrido[4,3-d]pyrimidin-6(5H)-yl)phenyl]cyclohexyl}acetic acid hydrochloride;
(trans-4-{4-[(7R)-4-amino-2-methoxy-7-methyl-5-oxo-7,8-dihydropyrido[4,3-d]pyrimidin-6(5H)-yl]phenyl}cyclohexyl)acetic acid;
(trans-4-{4-[4(7S)-4-amino-2-methoxy-7-methyl-5-oxo-7,8-dihydropyrido[4,3-d]pyrimidin-6(5H)-yl]phenyl}cyclohexyl)acetic acid;
{trans-4-[4-(4-amino-2-methoxy-5-oxo-7,8-dihydropyrido[4,3-d]pyrimidin-6(5H)-yl)-2-methylphenyl]cyclohexyl}acetic acid;
{trans-4-[4-(4-amino-2-methoxy-5-oxo-7,8-dihydropyrido[4,3-d]pyrimidin-6(5H)-yl)-2-chlorophenyl]cyclohexyl}acetic acid;
{trans-4-[4-(4-amino-2-methoxy-7-methyl-5-oxo-7,8-dihydropyrido[4,3-d]pyrimidin-6(5H)-yl)-2-fluorophenyl]cyclohexyl}acetic acid;
{trans-4-[4-(4-amino-2-methoxy-5-oxo-7,8-dihydropyrido[4,3-d]pyrimidin-6(5H)-yl)-2-fluorophenyl]cyclohexyl}acetic acid;
{trans-4-[4-(4-amino-2-isopropoxy-5-oxo-7,8-dihydropyrido[4,3-d]pyrimidin-6(5H)-yl)phenyl]cyclohexyl}acetic acid;
(trans-4-{4-[4-amino-2-(2-methoxyethoxy)-5-oxo-7,8-dihydropyrido[4,3-d]pyrimidin-6(5H)-yl]phenyl}cyclohexyl)acetic acid;
{trans-4-[4-(4-amino-2-ethoxy-5-oxo-7,8-dihydropyrido[4,3-d]pyrimidin-6(5H)-yl)phenyl]cyclohexyl}acetic acid;
{trans-4-[4-(4-amino-2-methoxy-7-methyl-5-oxo-7,8-dihydropyrido[4,3-d]pyrimidin-6(5H)-yl)phenyl]cyclohexyl}acetic acid;
{4-[4-(4-amino-2-methyl-5-oxo-7,8-dihydropyrido[4,3-d]pyrimidin-6(5H)-yl)phenyl]cyclohexyl}acetic acid;
{trans-4-[4-(4-amino-2-methyl-5-oxo-7,8-dihydropyrido[4,3-d]pyrimidin-6(5H)-yl)phenyl]cyclohexyl}acetic acid;
{cis-4-[4-(4-amino-2-methyl-5-oxo-7,8-dihydropyrido[4,3-d]pyrimidin-6(5H)-yl)phenyl]cyclohexyl}acetic acid;
{trans-4-[4-(4-amino-2,7-dimethyl-5-oxo-7,8-dihydropyrido[4,3-d]pyrimidin-6(5H)-yl)phenyl]cyclohexyl}acetic acid;
{trans-4-[4-(4-amino-2-ethyl-5-oxo-7,8-dihydropyrido[4,3-d]pyrimidin-6(5H)-yl)phenyl]cyclohexyl}acetic acid; and
{trans-4-[4-(4-amino-5-oxo-7,8-dihydropyrido[4,3-d]pyrimidin-6(5H)-yl)phenyl]cyclohexyl}acetic acid;
or a pharmaceutically acceptable salt thereof.

8. A pharmaceutical composition comprising (i) a compound of any one of the preceding claims or a pharmaceutically acceptable salt thereof; and (ii) a pharmaceutically acceptable excipient, diluent, or carrier.

9. The composition of claim 8 wherein said compound or said pharmaceutically acceptable salt thereof is present in a therapeutically effective amount.

10. The composition of claim 9 further comprising at least one additional pharmaceutical agent selected from the group consisting of an anti-obesity agent and an anti-diabetic agent.

11. The composition of claim 10 wherein said anti-obesity agent is selected from the group consisting of dirlotapide, mitratapide, implitapide, R56918 (CAS No. 403987), CAS No. 913541-47-6, lorcaserin, cetilistat, PYY₃₋₃₆, naltrexone, oleoyl-estrone, obinepitide, pramlintide, tesofensine, leptin, liraglutide, bromocriptine, orlistat, exenatide, AOD-9604 (CAS No. 221231-10-3) and sibutramine.

12. The composition of claim 10 wherein said anti-diabetic agent is selected from the group consisting of metformin, acetohexamide, chlorpropamide, diabinese, glibenclamide, glipizide, glyburide, glimepiride, gliclazide, glipentide, gliquidone, glisolamide, tolazamide, tolbutamide, tendamistat, trestatin, acarbose, adiposine, camiglibose, emiglitate, miglitol, voglibose, pradimicin-Q, salbostatin, balaglitazone, ciglitazone, darglitazone, englitazone, isaglitazone, pioglitazone, rosiglitazone, troglitazone, exendin-3, exendin-4, trodusquemine, reservatrol, hyrtiosal extract, sitagliptin, vildagliptin, alogliptin and saxagliptin.

13. A method for treating obesity and obesity-related disorders in animals comprising the step of administering to an animal in need of such treatment a therapeutically effective amount of a compound of any one of claims 1 through 7.

14. A method for treating or delaying the progression or onset of Type 2 diabetes and diabetes-related disorders in animals comprising the step of administering to an animal in need of such treatment a therapeutically effective amount of a compound of any one of claims 1 through 7.

15. A method for treating obesity and obesity-related disorders in animals comprising the step of administering to an animal in need of such treatment a pharmaceutical composition of any one of claims 8 through 12.

16. A method for treating or delaying the progression or onset of Type 2 diabetes and diabetes-related disorders in animals comprising the step of administering to an animal in need of such treatment a pharmaceutical composition of any one of claims 8 through 12.

17. A method for treating a disease, condition or disorder modulated by the inhibition of DGAT-1 in animals comprising the step of administering to an animal in need of such treatment two separate pharmaceutical compositions comprising
(i) a first composition comprising a compound of claim 1 through 7 or a pharmaceutically acceptable salt thereof, and a pharmaceutically acceptable excipient, diluent, or carrier; and
(ii) a second composition comprising at least one additional pharmaceutical agent selected from the group consisting of an anti-obesity agent and an anti-diabetic agent, and a pharmaceutically acceptable excipient, diluent, or carrier;
wherein said disease, condition or disorder modulated by the inhibition of DGAT-1 is selected from the group consisting of obesity, obesity-related disorders, Type 2 diabetes, and diabetes-related disorders.

18. The method of claim 17 wherein said anti-obesity agent is selected from the group consisting of dirlotapide, mitratapide, implitapide, R56918 (CAS No. 403987), CAS No. 913541-47-6, lorcaserin, cetilistat, PYY₃₋₃₆, naltrexone, oleoyl-estrone, obinepitide, pramlintide, tesofensine, leptin, liraglutide, bromocriptine, orlistat, exenatide, AOD-9604 (CAS No. 221231-10-3) and sibutramine; and
said anti-diabetic agent is selected form the group consisting of metformin, acetohexamide, chlorpropamide, diabinese, glibenclamide, glipizide, glyburide, glimepiride, gliclazide, glipentide, gliquidone, glisolamide, tolazamide, tolbutamide, tendamistat, trestatin, acarbose, adiposine, camiglibose, emiglitate, miglitol, voglibose, pradimicin-Q, salbostatin, balaglitazone, ciglitazone, darglitazone, englitazone, isaglitazone, pioglitazone, rosiglitazone, troglitazone, exendin-3, exendin-4, trodusquemine, reservatrol, hyrtiosal extract, sitagliptin, vildagliptin, alogliptin and saxagliptin.

19. The method of claim 17 or 18 wherein said first composition and said second composition are administered simultaneously.

20. The method of claim 17 or 18 wherein said first composition and said second composition are administered sequentially and in any order.

21. The use of a compound or a pharmaceutically acceptable salt thereof of claim 1 through 7 in the manufacture of a medicament for treating a disease, condition or disorder that is modulated by the inhibition of DGAT-1.
